(19) United States
(12) Patent Application Publication
(10) Pub. No.: US 2008/0206333 A1
(43) Pub. Date: Aug. 28, 2008

(54) Extended Release Pharmaceutical Formulations of S-Adenosylmethionine

(75) Inventor: Joshua Freedman, Santa Monica, CA (US)

Correspondence Address: WILSON SONSINI GOODRICH & ROSATI, 650 PAGE MILL ROAD, PALO ALTO, CA, 94304-1050, US

(73) Assignee: METHYLATION SCIENCES INTERNATIONAL SRL, Rockley (BB)

(21) Appl. No.: 12/024,059

(22) Filed: Jan. 31, 2008

Related U.S. Application Data
(60) Provisional application No. 60/887,565, filed on Jan. 31, 2007.

(51) Int. Cl. A61K 31/7076; A61P 1/16; A61P 25/00; A61P 25/30; A61P 25/32; A61P 25/36; A61K 9/107; A61K 9/28; A61K 9/22

(52) U.S. Cl. 424/468; 514/46; 424/474

(57) ABSTRACT

Extended release formulations of S-methyladenosylmethionine (SAMe) are provided, as are methods of treating various disorders using extended release SAMe formulations. The extended release formulations may be used to treat a variety of disorders, including liver disorders, psychiatric disorders and joint disorders. Thus, extended release SAMe formulations may be used to treat alcoholic liver disease, fatty liver disease, hepatitis, generalized anxiety disorder, obsessive compulsive disorder, post traumatic stress disorder, panic disorder, and depressive disorders such as depression (e.g. major clinical depression) and dysthymia.

### CROSS-REFERENCE TO RELATED APPLICATIONS AND CLAIM TO PRIORITY

This application claims priority to U.S. Provisional patent application Ser. No. 60/887,565, filed Jan. 31, 2007, which is incorporated herein by reference in its entirety.

### BACKGROUND OF THE INVENTION

S-adenosyl-L-methionine (“SAMe”) is a naturally occurring compound that is present in tissues throughout the body. At the molecular level, SAMe is involved in various metabolic pathways, including transmethylation, transsulfuration and aminopropylation (e.g. in the production of polyamines, such as spermidine and spermine, from putrescine). SAMe is thus involved in the biosynthesis of various hormones and neurotransmitters. Although the metabolic processes in which SAMe is involved occur throughout the body, most SAMe is produced in the liver.

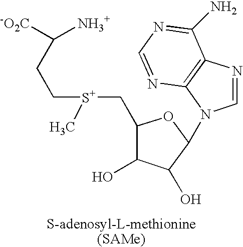

In the body, SAMe is synthesized from an amino acid, methionine, and a triphosphate nucleotide, ATP. In fact, aside from water, SAMe is considered the second most common metabolic molecule—ATP being the most common—in the body. Unfortunately, SAMe biosynthesis appears to decrease with age; and decreased SAMe production has been linked to aging, dementia, liver disease, alcoholism and depression. Indeed, SAMe has been subjected to numerous clinical trials for the treatment of various ailments, including arthritis, liver disease and depression.

SAMe supplementation was initially considered impractical, due to the instability of the SAMe ion during manufacturing, shipping and storage. Eventually stable salts of SAMe were developed (such as SAMe disulfate tosylate, the butanedisulfonate salt of SAMe, the di-p-toluene sulfonate disulfate of SAMe, the tri-p-toluene sulfonic acid salt of SAMe). Stable salts of SAMe are described in U.S. Pat. Nos. 3,954,726 and 4,057,686, each of which is incorporated herein by reference in its entirety. Numerous clinical trials have suggested the suitability of SAMe for treating a variety of conditions, such as liver disease, depression and arthritis. Enteric coated SAMe has been developed as a nutritional supplement for sale in the United States and other countries; and SAMe has also been available in Europe as a prescription drug for decades. However, the use of extended release SAMe has not heretofore been reported, nor has the use of extended release SAMe for the treatment of disease been previously reported.

### SUMMARY OF THE INVENTION

Some embodiments herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.95 when T is about 2 hours; Q is about 0.5 to about 1.0 when T is about 4 hours; Q is about 0.5 to about 1.0 when T is about 6 hours; Q is about 0.3 to about 0.9 when T is about 8 hours; and Q is about 0.15 to about 0.6 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.95 when T is about 2 hours; Q is about 0.5 to about 1.0 when T is about 4 hours; Q is about 0.5 to about 1.0 when T is about 6 hours; Q is about 0.3 to about 0.9 when T is about 8 hours; and Q is about 0.15 to about 0.6 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.95 when T is about 2 hours; Q is about 0.5 to about 1.0 when T is about 4 hours; Q is about 0.5 to about 1.0 when T is about 6 hours; Q is about 0.3 to about 0.9 when T is about 8 hours; and Q is about 0.15 to about 0.6 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the kit further comprises at least one dosage form selected from the group consisting of an immediate release SAMe dosage and an enterically coated immediate release SAMe dosage.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.6 to about 0.95 when T is about 2 hours; Q is about 0.65 to about 0.95 when T is about 4 hours; Q is about 0.9 to about 1.0 when T is about 6 hours; Q is about 0.7 to about 0.95 when T is about 8 hours; and Q is about 0.3 to about 0.65 (especially about 0.5 to about 0.6) when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.6 to about 0.95 when T is about 2 hours; Q is about 0.65 to about 0.95 when T is about 4 hours; Q is about 0.9 to about 1.0 when T is about 6 hours; Q is about 0.7 to about 0.95 when T is about 8 hours; and Q is about 0.3 to about 0.65 (especially about 0.5 to about 0.6) when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.6 to about 0.95 when T is about 2 hours; Q is about 0.65 to about 0.95 when T is about 4 hours; Q is about 0.9 to about 1.0 when T is about 6 hours; Q is about 0.7 to about 0.95 when T is about 8 hours; and Q is about 0.3 to about 0.65 (especially about 0.5 to about 0.6) when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the kit further comprises at least one dosage form selected from the group consisting of an immediate release SAMe dosage and an enterically coated immediate release SAMe dosage.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.9 to about 1.0 when T is about 4 hours; Q is about 0.3 to about 0.5 when T is about 8 hours; Q is about 0.2 to about 0.4 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.9 to about 1.0 when T is about 4 hours; Q is about 0.3 to about 0.5 when T is about 8 hours; Q is about 0.2 to about 0.4 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.9 to about 1.0 when T is about 4 hours; Q is about 0.3 to about 0.5 when T is about 8 hours; Q is about 0.2 to about 0.4 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the kit further comprises at least one dosage form selected from the group consisting of an immediate release SAMe dosage and an enterically coated immediate release SAMe dosage.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.7 to about 0.9 when T is about 2 hours; Q is about 0.7 to about 0.9 when T is about 4 hours; Q is about 0.9 to about 1.0 when T is about 6 hours; Q is about 0.4 to about 0.6 when T is about 8 hours; and Q is about 0.25 to about 0.45 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.7 to about 0.9 when T is about 2 hours; Q is about 0.7 to about 0.9 when T is about 4 hours; Q is about 0.9 to about 1.0 when T is about 6 hours; Q is about 0.4 to about 0.6 when T is about 8 hours; and Q is about 0.25 to about 0.45 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.7 to about 0.9 when T is about 2 hours; Q is about 0.7 to about 0.9 when T is about 4 hours; Q is about 0.9 to about 1.0 when T is about 6 hours; Q is about 0.4 to about 0.6 when T is about 8 hours; and Q is about 0.25 to about 0.45 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the kit further comprises at least one dosage form selected from the group consisting of an immediate release SAMe dosage and an enterically coated immediate release SAMe dosage.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.8 to about 1.0 when T is about 4 hours; Q is about 0.4 to about 0.8 when T is about 6 hours; Q is about 0.2 to about 0.7 when T is about 8 hours; and Q is about 0.2 to about 0.7 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.8 to about 1.0 when T is about 4 hours; Q is about 0.4 to about 0.8 when T is about 6 hours; Q is about 0.2 to about 0.7 when T is about 8 hours; and Q is about 0.2 to about 0.7 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.8 to about 1.0 when T is about 4 hours; Q is about 0.4 to about 0.8 when T is about 6 hours; Q is about 0.2 to about 0.7 when T is about 8 hours; and Q is about 0.2 to about 0.7 when T is about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the kit further comprises at least one dosage form selected from the group consisting of an immediate release SAMe dosage and an enterically coated immediate release SAMe dosage.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.5 to about 0.8 when T is about 2 hours; Q is about 0.8 to about 1.0 when T is about 4 hours; Q is about 0.8 to about 1.0 when T is about 6 hours; Q is about 0.3 to about 0.7 when T is about 8 hours; and Q is about 0.3 to about 0.7 when T is about 12 hours. In some embodiments Q is about 0.3 to about 0.7 at about 24 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.5 to about 0.8 when T is about 2 hours; Q is about 0.8 to about 1.0 when T is about 4 hours; Q is about 0.8 to about 1.0 when T is about 6 hours; Q is about 0.3 to about 0.7 when T is about 8 hours; and Q is about 0.3 to about 0.7 when T is about 12 hours. In some embodiments Q is about 0.3 to about 0.7 at about 24 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form×release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.5 to about 0.8 when T is about 2 hours; Q is about 0.8 to about 1.0 when T is about 4 hours; Q is about 0.8 to about 1.0 when T is about 6 hours; Q is about 0.3 to about 0.7 when T is about 8 hours; and Q is about 0.3 to about 0.7 when T is about 12 hours. In some embodiments Q is about 0.3 to about 0.7 at about 24 hours. In some embodiments, the kit further comprises at least one dosage form selected from the group consisting of an immediate release SAMe dosage and an enterically coated immediate release SAMe dosage. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.5 to about 0.7 when T is about 4 hours; Q is about 0.6 to about 0.8 when T is about 6 hours; Q is about 0.8 to about 1.0 when T is about 8 hours; and Q is about 0.5 to about 0.7 when T is about 12 hours. In some embodiments Q is about 0.5 to about 0.7 at about 24 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.5 to about 0.7 when T is about 4 hours; Q is about 0.6 to about 0.8 when T is about 6 hours; Q is about 0.8 to about 1.0 when T is about 8 hours; and Q is about 0.5 to about 0.7 when T is about 12 hours. In some embodiments Q is about 0.5 to about 0.7 at about 24 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form×release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.5 to about 0.7 when T is about 4 hours; Q is about 0.6 to about 0.8 when T is about 6 hours; Q is about 0.8 to about 1.0 when T is about 8 hours; and Q is about 0.5 to about 0.7 when T is about 12 hours. In some embodiments Q is about 0.5 to about 0.7 at about 24 hours. In some embodiments, the kit further comprises at least one dosage form selected from the group consisting of an immediate release SAMe dosage and an enterically coated immediate release SAMe dosage. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease.

Some embodiments described herein provide a method of treating a disorder selected from the group consisting of osteoarthritis, rheumatoid arthritis fibromyalgia, a psychiatric disorder, an inflammatory condition, a central nervous system (CNS) disorder, a pain disorder and a liver disorder in a patient, comprising administering to the patient an extended release dosage comprising a S-adenosyl methionine (SAMe), or a pharmaceutically acceptable salt thereof, wherein the extended release dosage provides a blood plasma concentration versus time curve for SAMe in a patient population as follows: blood plasma concentration of SAMe of 0 to 200 nmol/L at about 2 hours, blood plasma concentration of SAMe of about 100 to 400 nmol/L at about 4 hours, and a SAMe Cₘₐₓ of from 100 to 400 nmol/L that occurs at a time Tₘₐₓ at least about 4 hours after administration of the extended release dosage. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ a is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a S-adenosyl methionine (SAMe), or a pharmaceutically acceptable salt thereof, wherein the extended release dosage provides a blood plasma concentration versus time curve for SAMe in a patient population as follows: blood plasma concentration of SAMe of 0 to 200 nmol/L at about 2 hours, blood plasma concentration of SAMe of about 100 to 400 nmol/L at about 4 hours, and a SAMe Cₘₐₓ of from 100 to 400 nmol/L that occurs at a time Tₘₐₓ at least about 4 hours after administration of the extended release dosage.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a S-adenosyl methionine (SAMe), or a pharmaceutically acceptable salt thereof, wherein the extended release dosage provides a blood plasma concentration versus time curve for SAMe in a patient population as follows: blood plasma concentration of SAMe of 0 to 200 nmol/L at about 2 hours, blood plasma concentration of SAMe of about 100 to 400 nmol/L at about 4 hours, and a SAMe Cₘₐₓ of from 100 to 400 nmol/L that occurs at a time Tₘₐₓ at least about 4 hours after administration of the extended release dosage. In some embodiments, the kit further comprises at least one dosage form selected from the group consisting of an immediate release SAMe dosage and an enterically coated immediate release SAMe dosage.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=[SAMe]_{T}/Cₘₐₓ in blood plasma at time T after administration of the extended release dosage as follows: Q is 0 to about 1.0 at time T of about 4 hours, Q is about 0.5 to about 1.0 at time T about 8 hours, and Q is about 0.5 to about 0.8 at time T of about 12 hours. In some embodiments, the disorder is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein the extended release dosage provides a quotient Q=[SAMe]_{T}/Cₘₐₓ in blood plasma at time T after administration of the extended release dosage as follows: Q is 0 to about 1.0 at time T of about 4 hours, Q is about 0.5 to about 1.0 at time T about 8 hours, and Q is about 0.5 to about 0.8 at time T of about 12 hours.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=[SAMe]_{T}/Cₘₐₓ in blood plasma at time T after administration of the extended release dosage as follows: Q is 0 to about 1.0 at time T of about 4 hours, Q is about 0.5 to about 1.0 at time T about 8 hours, and Q is about 0.5 to about 0.8 at time T of about 12 hours. In some embodiments, the kit further comprises at least one dosage form selected from the group consisting of an immediate release SAMe dosage and an enterically coated immediate release SAMe dosage.

Some embodiments described herein provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein blood plasma concentrations of SAMe ([SAMe]_{T}, wherein T is a time after administration of the SAMe to a patient population) provided by the extended release dosage, at time points T of about 2 hours, about 4 hours, about 6 hours and about 8 hours after administration of the extended release dosage to the patient, are about 40 to 100 percent of the C_{Max}. In some embodiments, the disorder is a liver disorder selected from the group consisting of alcoholic liver disease, fatty liver disease and hepatitis. In some embodiments, the disorder is an, inflammatory disorder such as inflammatory bowel disease (IBD), Crohn's disease or ulcerative colitis (UC). In some embodiments, the disorder is a psychiatric disorder selected from the group consisting of depressive disorders, eating disorders, bipolar disorder, abuse disorders, dependence disorders, Axis II disorders, psychosis and anxiety disorders. In some embodiments, the psychiatric disorder is an anxiety disorder selected from the group consisting of generalized anxiety disorder, post traumatic stress disorder, panic disorder and obsessive compulsive disorder. In some embodiments, the psychiatric disorder is a depressive disorder. In some embodiments, the depressive disorder is major depressive disorder, minor depression, brief recurrent depression, dysthymia or depression NOS. In some embodiments, the psychiatric disorder is an eating disorder selected from the group consisting of bulimia nervosa, anorexia nervosa, binge eating disorder, obesity, or eating disorder NOS. In some embodiments, the psychiatric disorder is bipolar disorder, an abuse disorder or a dependence disorder. In some embodiments, the psychiatric disorder includes abuse of, or dependence on, alcohol, cocaine, codeine, oxycodone, hydrocodone or other opiates. In some embodiments, the psychiatric disorder is an Axis II disorder selected from borderline personality disorder. In some embodiments, the disorder is a CNS disorder such as Parkinson's syndrome or Alzheimer's disease. In some embodiments, the Tₘₐₓ is at least about 6 hours after administration of the extended release dosage. In some embodiments, the Tₘₐₓ is about 4 to about 12 hours after administration of the extended release dosage. In some embodiments, the dose is administered in 1 to 4, 1 to 5 or 1 to 6 discrete dosage units. In some embodiments, the patient is fed prior to administration of the SAMe. In some embodiments, the method further comprises administering to the patient one or more additional active compounds. In some embodiments, the one or more additional compounds comprise vitamin B12 (B12), folate (folic acid or a biologically acceptable salt thereof), or both. In some embodiments, at least a portion of the SAMe is contained within an extended release matrix, an osmotic extended release core or a pulsatile release formulation.

Some embodiments described herein provide an extended release dosage comprising a therapeutically effective amount of SAMe, wherein blood plasma concentrations of SAMe ([SAMe]_{T}, wherein T is a time after administration of the SAMe to a patient population) provided by the extended release dosage, at time points T of about 2 hours, about 4 hours, about 6 hours and about 8 hours after administration of the extended release dosage to the patient, are about 40 to 100 percent of the C_{Max}. In some embodiments, the dosage comprises a monolithic extended release core. In some embodiments, the dosage comprises a monolithic extended release core and an extended release coating. In some embodiments, the extended release coating comprises a pore former.

Some embodiments described herein provide a kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein blood plasma concentrations of SAMe ([SAMe]_{T}, wherein T is a time after administration of the SAMe to a patient population) provided by the extended release dosage, at time points T of about 2 hours, about 4 hours, about 6 hours and about 8 hours after administration of the extended release dosage to the patient, are about 40 to 100 percent of the C_{Max}. In some embodiments, the kit further comprises at least one dosage form selected from the group consisting of an immediate release SAMe dosage and an enterically coated immediate release SAMe dosage.

Some embodiments described herein provide an extended release, oral dosage for administration of SAMe to a patient, comprising a therapeutically effective amount of SAMe, wherein dissolution of the oral dosage in a USP II dissolution apparatus in aqueous buffer having an initial pH of about 6.8 provides less than about 70% release of SAMe after about 2 hours, less than about 80% release of SAMe after about 3 hours and less than about 100% release of SAMe after about 4 hours.

Some embodiments described herein provide an extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours.

Some embodiments described herein provide an extended release, oral dosage for administration of SAMe to a patient, comprising a therapeutically effective amount of SAMe, wherein dissolution of the oral dosage in a USP II dissolution apparatus in aqueous buffer at an initial pH of about 6.8 provides less about 70% release of SAMe after about 2 hours, less than about 80% release of SAMe after about 3 hours, less than about 100% release of SAMe after about 4 hours, and at least about 50% release after about 8 hours.

Some embodiments described herein provide an extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours, and at least about 70% release after about 8 hours.

Some embodiments described herein provide an extended release, oral dosage for administration of SAMe to a patient, comprising a therapeutically effective amount of SAMe, liquid paraffin, magnesium aluminometasilicate and 0-6% of an extended release coating, which optionally comprises a pore former.

Some embodiments described herein provide a kit for administration of SAMe to a patient, comprising at least a first dosage form and a second dosage form, wherein said first dosage form is an immediate release dosage optionally comprising an enteric coating; and the second dosage form is an extended release dosage form. In some embodiments, the kit comprises an extended release, oral dosage for administration of SAMe to a patient, comprising a therapeutically effective amount of SAMe, wherein dissolution of the oral dosage in a USP II dissolution apparatus in aqueous buffer having an initial pH of about 6.8 provides less about 70% release of SAMe after about 2 hours, less than about 80% release of SAMe after about 3 hours and less than about 100% release of SAMe after about 4 hours. In some embodiments, the kit comprises an extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours. In some embodiments, the kit comprises an extended release, oral dosage for administration of SAMe to a patient, comprising a therapeutically effective amount of SAMe, wherein dissolution of the oral dosage in a USP II dissolution apparatus in aqueous buffer at an initial pH of about 6.8 provides less about 70% release of SAMe after about 2 hours, less than about 80% release of SAMe after about 3 hours, less than about 100% release of SAMe after about 4 hours, and at least about 50% release after about 8 hours. In some embodiments, the kit comprises an extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours, and at least about 70% release after about 8 hours. In some embodiments, the kit comprises an extended release, oral dosage for administration of SAMe to a patient, comprising a therapeutically effective amount of SAMe, liquid paraffin, magnesium aluminometasilicate and 0-6% of an extended release coating, which optionally comprises a pore former.

Given the promising therapeutic profile of SAMe, it is considered that an extended release formulation of SAMe would provide advantageous pharmacokinetic properties for the use of SAMe in the treatment of a variety of psychiatric, neurological and other medical conditions, symptoms and disease states. However, as noted above, extended release SAMe has not been previously reported. There is thus a need for extended release formulations of SAMe, as well as therapeutic methods of using the extended release formulations for the treatment of one or more psychiatric or neurological conditions, such as depression. Embodiments of the present invention address this need and provide related advantages as well.

The foregoing and further objects are addressed by embodiments of the present invention, which provide a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder in a patient, comprising administering to the patient an extended release dosage comprising a therapeutically effective amount of SAMe. In some embodiments, the extended release dosage provides a blood plasma concentration of SAMe as follows: 0 to 200 nmol/L from 0 to 2 hours, 200 to 1000 nmol/L from 2 to 4 hours, and a Cmax of from 300 to 2000 nmol/L that occurs at a time Tmax at least about 4 hours after administration of the extended release dosage. In some specific embodiments of the invention, Tmax is at least about 7 hours after administration of the extended release dosage. In some embodiments, Tmax is about 5 to about 12 hours after administration of the extended release dosage. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression not otherwise specified (depression NOS). In other embodiments, the psychiatric disorder to be treated is an eating disorder selected from the group consisting of bulimia nervosa, anorexia nervosa, binge eating disorder, obesity, or eating disorder not otherwise specified (eating disorder NOS). In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some embodiments the patient may be fasted prior to administration of the therapeutically effective amount of extended release SAMe. In other embodiments, the patient may be fed prior to administration of the therapeutically effective amount of SAMe.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder in a patient, comprising administering to the patient an extended release dosage comprising a therapeutically effective amount of SAMe. In some embodiments, the extended release dosage provides a ratio [SAMe]/[SAMe]max in blood plasma after administration of the extended release dosage as follows: 0 to 0.95 from 0 to 4 hours, 0.25 to 1.0 from 4 to 8 hours, and 0.25 to 1.0 from 8 to 12 hours after administration of the extended release dosage. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression NOS. In other embodiments, the psychiatric disorder to be treated is an eating disorder selected from the group consisting of bulimia nervosa, anorexia nervosa, binge eating disorder, obesity, or eating disorder NOS. In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some embodiments the patient may be fasted prior to administration of the therapeutically effective amount of extended release SAMe. In other embodiments, the patient may be fed prior to administration of the therapeutically effective amount of SAMe.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder in a patient, comprising administering to the patient an extended release dosage comprising a therapeutically effective amount of SAMe. In some embodiments, the dosage provides: approximately 0 to 60 percent of the therapeutically effective amount 0 to 4 hours after administration, approximately 20 to 80 percent of the therapeutically effective amount 4 to 8 hours after administration, and approximately 30 to 100 percent of the therapeutically effective amount 8 to 36 (e.g. about 8 to 12 or 8 to 24) hours after administration. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression NOS. In other embodiments, the psychiatric disorder to be treated is an eating disorder selected from the group consisting of bulimia nervosa, anorexia nervosa, binge eating disorder, obesity, or eating disorder NOS. In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some embodiments the patient may be fasted prior to administration of the therapeutically effective amount of extended release SAMe. In other embodiments, the patient may be fed prior to administration of the therapeutically effective amount of SAMe.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder in a patient, comprising administering to the patient an extended release dosage comprising a therapeutically effective amount of SAMe. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression NOS. In other embodiments, the psychiatric disorder to be treated is an eating disorder selected from the group consisting of bulimia nervosa, anorexia nervosa, binge eating disorder, obesity, or eating disorder NOS. In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some embodiments the patient may be fasted prior to administration of the therapeutically effective amount of extended release SAMe. In other embodiments, the patient may be fed prior to administration of the therapeutically effective amount of SAMe.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder in a patient, comprising administering to the patient an extended release dosage comprising a therapeutically effective amount of SAMe. In some such embodiments, the blood plasma concentrations of SAMe provided by the extended release dosage, over a period of from 0 to 24 hours after administration of the extended release dosage to the patient, are approximate 15 to 85 percent of the Max for a non-extended release formulation of SAMe. In some such embodiments, the CMax of SAMe provided by the extended release dosage is in the range of about 15 to about 55 percent of the CMax for a non-extended release formulation of SAMe. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression NOS. In other embodiments, the psychiatric disorder to be treated is an eating disorder selected from the group consisting of bulimia nervosa, anorexia nervosa, binge eating disorder, obesity, or eating disorder NOS. In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some embodiments the patient may be fasted prior to administration of the therapeutically effective amount of extended release SAMe. In other embodiments, the patient may be fed prior to administration of the therapeutically effective amount of SAMe.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder in a patient, comprising administering to the patient a therapeutically effective amount of SAMe. In some such embodiments, the method provides approximately 0 to 60 percent of the therapeutically effective amount of SAMe (AUC) 0 to 4 hours after administration, approximately 20 to 80 percent of the therapeutically effective amount of SAMe 4 to 8 hours after administration, and approximately 25 to 100 percent of the therapeutically effective amount SAMe 8 to 36 (e.g. 8 to 12 or 8 to 24) hours after administration. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression NOS. In other embodiments, the psychiatric disorder to be treated is an eating disorder selected from the group consisting of bulimia nervosa, anorexia nervosa, binge eating disorder, obesity, or eating disorder NOS. In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some specific embodiments, the depression ameliorating effect is produced in the patient for a period of at least about 24 hours after administration of the SAMe to the patient.

In some embodiments, the invention provides an extended release dosage for the treatment of a disorder, comprising a therapeutically effective amount of SAMe, wherein the dosage provides 0 to 60 percent of the therapeutically effective amount (AUC) 0 to 4 hours after administration to a subject, approximately 20 to 80 percent of the therapeutically effective amount 4 to 8 hours after administration to the subject, and approximately 25 to 100 percent of the therapeutically effective amount 8 to 36 (e.g. 8 to 12 or 8 to 24) hours after administration to the subject. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression NOS. In other embodiments, the psychiatric disorder to be treated is an eating disorder selected from the group consisting of bulimia nervosa, anorexia nervosa, binge eating disorder, obesity, or eating disorder NOS. In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some embodiments the patient may be fasted prior to administration of the therapeutically effective amount of extended release SAMe. In other embodiments, the patient may be fed prior to administration of the therapeutically effective amount of SAMe.

In some embodiments, the invention provides an extended release dosage for the treatment of a disorder, comprising a therapeutically effective amount of SAMe, wherein the dosage provides an in vitro extended release profile in an aqueous solution wherein: 0 to 60 percent of the therapeutically effective amount is released into the aqueous solution 0 to 4 hours after introduction of the extended release dosage to the aqueous solution, approximately 20 to 80 percent of the therapeutically effective amount is released into the aqueous solution 4 to 8 hours after introduction of the extended release dosage to the aqueous solution, and approximately 25 to 100 percent of the therapeutically effective amount is released into the aqueous solution 8 to 36 (e.g. 8 to 12 or 8 to 24) hours after introduction of the extended release dosage to the aqueous solution. In some embodiments, the disorder to be treated is a liver disorder selected from the group consisting of alcoholic liver disease, fatty liver disease and hepatitis. In some embodiments, the disorder is an inflammatory disorder such as inflammatory bowel disease (IBD), Crohn's disease or ulcerative colitis (UC). In some embodiments, the disorder to be treated is a psychiatric disorder selected from the group consisting of depressive disorders, eating disorders, bipolar disorder, abuse disorders, dependence disorders, Axis II disorders, psychosis and anxiety disorders. In some particular embodiments, the psychiatric disorder to be treated is an anxiety disorder selected from the group consisting of generalized anxiety disorder, post traumatic stress disorder, panic disorder and obsessive compulsive disorder. In some particular embodiments, the psychiatric disorder to be treated is a depressive disorder. Some more particular embodiments, the depressive disorder is major depressive disorder, minor depression, brief recurrent depression, dysthymia or depression NOS. In other embodiments, the psychiatric disorder to be treated is an eating disorder selected from the group consisting of bulimia nervosa, anorexia nervosa, binge eating disorder, obesity, or eating disorder NOS. In some embodiments, the psychiatric disorder to be treated is bipolar disorder, an abuse disorder or a dependence disorder. In some particular embodiments, the psychiatric disorder to be treated includes abuse of, or dependence on, alcohol, cocaine, codeine, oxycodone, hydrocodone or other opiates. In some embodiments the patient may be fasted prior to administration of the therapeutically effective amount of extended release SAMe. In other embodiments, the patient may be fed prior to administration of the therapeutically effective amount of SAMe.

It is contemplated that extended release S-adenosylmethionine (as compared to immediate release SAMe) may be characterized by a more rapid onset of action and thus may reduce the risk of suicidal behavior, suicide attempts or successful suicide in psychiatric patients, by increasing the rate of response to SAMe therapy. In addition, it is contemplated that treatment with extended release SAMe may be characterized by decreased side effects, especially gastrointestinal side effects normally associated with high doses of SAMe. Thus, treatment of psychiatric conditions with extended release SAMe according to the present invention may result in a reduction in suffering and a more rapid improvement in functioning.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph comparing dissolution profiles of SAMe monolithic cores coated with ethylcellulose/pore former coating (70:30 and 80:20 of polymer:pore former ratio).

FIG. 2 is a graph comparing dissolution profiles of tablets coated with ethylcellulose polymer mixed with pore former in ratios of 70:30 and 60:40 (polymer:pore former.)

FIG. 3 is a graph comparing the dissolution profiles of various prototype extended release SAMe tablets in pH 6.8 buffer and 1 N HCl.

FIG. 4 is a graph showing dissolution profiles of monolithic extended release tablets coated with ethylcellulose 60:40 with 2.0%, 2.5%, and 4.0% in 0.1 N HCl.

FIG. 5 is a graph showing the plasma concentration versus time plots for immediate release, enteric coated SAMe. Each patient was administered 4×400 mg tablets (1600 mg total) of SAMe.

FIG. 6 is a graph showing plasma concentration versus time for a monolithic extended release core (0% coated), and 2%, 4%, and 6% coated monolithic cores, wherein the coating is ethylcellulose mixed with pore former in a ration of ethylcellulose to pore former of 60:40. Each patient was administered 4×400 mg tablets (1600 mg total) of SAMe.

FIG. 7 is a graph showing the plasma concentration versus time plots for immediate release, enteric coated SAMe, a monolithic extended release core (0% coated), and 2%, 4%, and 6% coated monolithic cores, wherein the coating is ethylcellulose mixed with pore former in a ration of ethylcellulose to pore former of 60:40. Each patient was administered 4×400 mg tablets (1600 mg total) of SAMe.

FIG. 8 is a graphical comparison of area under the plasma concentration (AUC) calculations for immediate release, enteric coated SAMe, a monolithic extended release core (0% coated), and 2%, 4%, and 6% coated monolithic cores, wherein the coating is ethylcellulose mixed with pore former in a ration of ethylcellulose to pore former of 60:40.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention is directed to extended release formulations of SAMe and methods of using the same, e.g. for the treatment of depression in a once-a-day (q.d.) formulation. As used herein the term “SAMe” refers to S-adenosyl-L-methionine (or, more simply, “S-adenosylmethionine”). As can be seen in the structural formula above, SAMe appears as a charged species, having two positive and one negative center in physiologic solution. In its solid form, SAMe is always present as a salt. While the net charge of SAMe would suggest that it could form a salt with a single, negatively charged species, such as chloride, it is more common to find SAMe in a stable salt form, e.g. with p-toluenesulfonic acid as the negative counter ion, alone or in combination with one or more additional salt-forming substances (e.g. mineral or organic acids and/or amino acids). (See U.S. Pat. No. 3,893,999, incorporated herein by reference in its entirety). Other stable SAMe salts are described in, for example, U.S. Pat. No. 5,128,249, which teaches particular stable salts of SAMe. Thus, as used herein SAMe refers both to the stable salts of SAMe and to the ionic form of SAMe when present in vivo. When a mass, weight, concentration (e.g. wt.-%) or other mass-dependent unit (that is a unit of measurement that includes mass of SAMe in the numerator or denominator) is used in reference to SAMe herein, unless otherwise specified, it relates to the mass of the SAMe cation exclusive of the counter-anion. Where the mass of the SAMe salt in intended, this is specifically stated.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder in a patient, comprising administering to the patient an extended release dosage comprising a therapeutically effective amount of SAMe, wherein the extended release dosage provides a blood plasma concentration of SAMe as follows: 0 to 200 nmol/L from 0 to 2 hours, 200 to 1000 nmol/L from 2 to 4 hours, and a Cmax of from 300 to 2000 nmol/L that occurs at a time Tmax at least about 4 hours after administration of the extended release dosage. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia. In some embodiments, the Tmax is at least about 7 hours after administration of the extended release dosage. In some embodiments, Tmax is about 4 to about 12 hours after administration of the extended release dosage.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a ratio [SAMe]/[SAMe]max in blood plasma after administration of the extended release dosage as follows: 0 to 0.95 from 0 to 4 hours, 0.23 to 1.0 from 4 to 8 hours, and 0.25 to 1.0 from 8 and 12 hours after administration of the extended release dosage. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the dosage provides: approximately 0 to 60 percent of the therapeutically effective amount 0 to 4 hours after administration, approximately 20 to 80 percent of the therapeutically effective amount 4 to 8 hours after administration, and approximately 30 to 100 percent of the therapeutically effective amount 8 to 36 (e.g. about 8 to 12 or 8 to 24) hours after administration. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein blood plasma concentrations of SAMe provided by the extended release dosage, over a period of from 0 to 24 hours after administration of the extended release dosage to the patient, are approximate 15 to 85 percent of the CMax for a non-extended release formulation of SAMe. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder wherein the CMax of SAMe provided by the extended release dosage is in the range of about 15 to about 55 percent of the CMax for a non-extended release formulation of SAMe. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia.

In some embodiments, the invention provides a method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient a therapeutically effective amount of SAMe by providing: approximately 0 to 60 percent of the therapeutically effective amount of SAMe 0 to 4 hours after administration, approximately 20 to 80 percent of the therapeutically effective amount of SAMe 4 to 8 hours after administration, and approximately 30 to 100 percent of the therapeutically effective amount SAMe 8 to 36 (e.g. about 8 to 12 or 8 to 24) hours after administration. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia.

In some embodiments, the invention provides an extended release dosage for the treatment of a disorder in a patient, comprising a therapeutically effective amount of SAMe, wherein the dosage provides 0 to 60 percent of the therapeutically effective amount (AUC) 0 to 4 hours after administration to a subject, approximately 20 to 80 percent of the therapeutically effective amount 4 to 8 hours after administration to the subject, and approximately 25 to 100 percent of the therapeutically effective amount 8 to 36 (e.g. 8 to 12 or 8 to 24) hours after administration to the subject. The extended release SAMe dosage is useful for treating a variety of disorders, such as osteoarthritis, rheumatoid arthritis, fibromyalgia, psychiatric disorders, pain disorders and liver disorders. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia.

In some embodiments, the invention provides an extended release dosage for the treatment of a disorder in a patient, comprising a therapeutically effective amount of SAMe, wherein the dosage provides an in vitro extended release profile in an aqueous solution wherein: 0 to 60 percent of the therapeutically effective amount is released into the aqueous solution 0 to 4 hours after introduction of the extended release dosage to the aqueous solution, approximately 20 to 80 percent of the therapeutically effective amount is released into the aqueous solution 4 to 8 hours after introduction of the extended release dosage to the aqueous solution, and approximately 25 to 100 percent of the therapeutically effective amount is released into the aqueous solution 8 to 36 (e.g. about 8 to 12 or 8 to 24) hours after introduction of the extended release dosage to the aqueous solution. In some embodiments, the disorder is selected from the group consisting of fibromyalgia, psychiatric disorders (such as depressive disorders and anxiety disorders), pain disorders and liver disorders. In some embodiments, the disorder is a liver disorder selected from alcoholic liver disease, fatty liver or hepatitis. In some embodiments, the disorder is a psychiatric disorder selected from depressive disorders (e.g. depression or dysthymia) and anxiety disorders. In some more specific embodiments, the disorder is an anxiety disorder selected from generalized anxiety disorder, post traumatic stress disorder, panic disorder and obsessive compulsive disorder. In some specific embodiments, the psychiatric disorder is a depressive disorder, such as depression (e.g. major clinical depression) or dysthymia.

Disorders and Diseases to be Treated with Extended Release Same

The extended release SAMe formulations of the present invention are expected to provide relief from one or more symptoms of a variety of physiological disorders and disease states, such as fibromyalgia, psychiatric disorders, pain disorders and a liver disorders. Among the psychiatric disorders expected to respond favorably to extended release SAMe treatment, there may be mentioned depression (e.g. clinical depression or dysthymia) and anxiety disorders. Among the anxiety disorders that are expected to respond positively to extended release SAMe therapy include generalized anxiety disorder, post traumatic stress disorder, panic disorder or obsessive compulsive disorder. Among the liver disorders that are expected to respond positively to extended release SAMe therapy include alcoholic liver disease, fatty liver disease (non-alcoholic) and hepatitis (both viral and non-viral). Among the advantages provided by extended release SAMe formulations of the invention, there may be mentioned the convenience and concomitant improved patient compliance due to once-a-day dosing, an improved side-effect profile (such as decreased stomach irritation and potentially decreased tendency to induce mania in manic depressive patients or patients at risk for manic episodes) and other side effects associate with or caused by the relatively high doses of SAMe (typically about 400 to about 3200 mg/day, more typically about 800 to about 1600 mg/day) necessary to achieve a therapeutic effect.

As used herein, the term “therapeutic effect” and its grammatical variants (e.g. “therapeutically effective”) includes ameliorating at least one symptom of a physiological disorder or disease state in a patient, typically a human patient, and more typically an adult human patient (although in some embodiments human pediatric patients are not excluded). Various symptoms of specific physiological disorders and disease states which are contemplated as being treatable within the context of the present invention are set forth in detail below. However, it is to be recognized that the understanding of various disease states by those of skill in the art is not static. Thus, though the following description is intended to be illustrative of the various disorders, disease states and symptoms that may be treated using the extended release SAMe formulations according to the present invention, the person skilled in the art will be expected to apply such knowledge as is generally possessed by the skilled clinician in diagnosing and treating specific disorders and disease states with the extended release SAMe formulations of the invention. In particular, unless otherwise specified, a symptom that one of skill in the art would normally associate with one of the enumerated disorders and disease states is not excluded from the present disclosure merely because it is not specifically mentioned herein.

Osteoarthritis

Osteoarthritis (OA), is a condition in which low-grade inflammation results in pain in the joints, caused by wearing of the cartilage that covers and acts as a cushion inside joints. As the bone surfaces become less well protected by cartilage, the patient experiences pain upon weight bearing, including walking and standing. Due to decreased movement because of the pain, regional muscles may atrophy, and ligaments may become more lax. OA is the most common form of arthritis. Although the word ‘osteoarthritis’ literally suggests inflammation of the joints formed by adjacent bones, OA need not be characterized by inflammation.

The main symptom of OA is chronic pain, causing loss of mobility and often stiffness. OA-associated pain is generally described as a sharp ache, or a burning sensation in the associated muscles and tendons. OA can cause a crackling noise (called “crepitus”) when the affected joint is moved or touched; and patients may experience muscle spasm and contractions in the tendons. Occasionally, the joints may also be filled with fluid. Humid weather (especially cold, humid weather) increases the pain in many patients.

OA commonly affects the hand, feet, spine, and the large weight-bearing joints, such as the hips and knees, although in theory, any joint in the body can be affected. As OA progresses, the affected joints appear larger, are stiff and painful, and usually feel worse, the more they are used throughout the day, thus distinguishing it from rheumatoid arthritis.

In smaller joints, such as at the fingers, hard bony enlargements, called Heberden's nodes (on the distal interphalangeal joints) and/or Bouchard's nodes (on the proximal interphalangeal joints), may form, and though they are not necessarily painful, they do limit the movement of the fingers significantly. OA at the toes leads to the formation of bunions, rendering them red or swollen.

SAMe has been marketed as a nutritional supplement for the treatment of osteoarthritis and several clinical trials have been completed, in which it has been found that SAMe is an effective therapeutic agent for the treatment of OA. Thus, the present invention contemplates treatment of OA using an extended release SAMe formulation of the present invention. As SAMe has been shown to induce chondrocyte-mediated production of new cartilage, it is contemplated that extended release SAMe of the invention may be useful in the treatment of rheumatoid arthritis and other disorders and diseases affecting the joints. Whereas aspirin and other non-steroidal anti inflammatory drugs (NSAIDs) tend to suppress proteoglycan synthesis, and thus inhibit production of new cartilage and synovial fluid, SAMe has the opposite effect. Moreover, whereas NSAIDs have negative gastrointestinal effects in some patients, SAMe has been shown to have some gastrointestinal protective effects. Thus, the extended release SAMe formulations of the present invention are expected to be useful either in the palliation of the negative effects of aspirin, ibuprofen or other NSAID, or in the prevention of such negative effects, either in serial or combination therapy. Consequently, in some embodiments of the invention, the extended release SAMe compositions may include a therapeutically effective amount of an NSAID drug, such as aspirin or ibuprofen, for the treatment of osteoarthritis or other joint disorder. In other embodiments, SAMe may be co-administered with one or more doses of NSAID to treat osteoarthritis or another joint disorder.

SAMe has proven effective in the treatment of osteoarthritis and other joint diseases in clinical trials. Thus, it is expected that the extended release SAMe formulations of the invention will also be effective in treating osteoarthritis and other joint diseases. Contemplated dosages of extended release SAMe formulations for the treatment of osteoarthritis and other joint diseases are from about 400 to about 3200 mg/day given on a once a day (or at most twice a day) basis.

Psychiatric Disorders

Psychiatric disorders (depressive disorders or anxiety disorders): A number of psychiatric and psychological conditions have been identified, which are contemplated as being amenable to treatment with the extended release SAMe formulations of the present invention. Among these, depression is a currently preferred indication; however other indications, especially dysthymia, generalized anxiety disorder, post traumatic stress disorder, panic disorder and obsessive compulsive disorder, are contemplated as indications for the extended release SAMe formulations according to the present invention. The symptoms and diagnosis of each of these disorders is discussed in more detail below. It is thus expected that the person skilled in the art will be able to treat one or more psychiatric disorder with the extended release SAMe formulations according to the invention. It is contemplated that doses of about 400 to about 3200 mg/day of SAMe, given on a once a day basis (or at most twice a day), will provide therapeutic benefit to a patient suffering from a psychiatric disorder, such as a depressive disorder (e.g. clinical depression or dysthymia) or an anxiety disorder (such as generalized anxiety disorder, post traumatic stress disorder, panic disorder and obsessive compulsive disorder). In some currently preferred embodiments, the dose of extended release SAMe is about 800 to about 1600 mg/day, given on a once a day basis.

Depressive Disorders

Depressive disorders can include clinical depression (e.g. major clinical depression) and dysthymia. These disorders are discussed in more detail below. It is contemplated that doses of about 400 to about 3200 mg/day of SAMe, given on a once a day basis (or at most twice a day), will provide therapeutic benefit to a patient suffering from a depressive disorders, such as clinical depression and dysthymia. In some currently preferred embodiments, the dose of extended release SAMe is about 800 to about 1600 mg/day, given on a once a day basis.

Depression (Clinical Depression; Major Clinical Depression)

Clinical depression is a common psychiatric disorder. In general, clinical depression is a feeling melancholia or sadness of such severity and/or duration that it negatively affects the patient's social functioning. Clinical depression can vary in severity and duration. A high percentage of persons in the general population reports the symptoms of clinical depression at least once in their lifetime. According to the Diagnostic and Statistical Manual of Mental Disorders, Fourth Edition (DSM-IV-TR) a major depressive episode is diagnosed when at least five of the following symptoms have been present during the same 2-week period and represent a change from previous functioning; and at least one of the symptoms is depressed mood, loss of interest or loss of pleasure (anhedonia): (1) depressed mood most of the day, nearly every day; (2) markedly diminished interest or pleasure in all, or almost all, activities most of the day, nearly every day; (3) significant weight loss when not dieting or weight gain (e.g., a change of more than 5% of body weight in a month), or decrease or increase in appetite nearly every day; (4) insomnia or hypersomnia nearly every day; (5) psychomotor agitation or retardation nearly every day; (6) fatigue or loss of energy nearly every day; (7) feelings of worthlessness or excessive or inappropriate guilt (which may be delusional) nearly every day; (8) diminished ability to think or concentrate, or indecisiveness, nearly every day; (9) recurrent thoughts of death (not just fear of dying), recurrent suicidal ideation without a specific plan, or a suicide attempt or a specific plan for committing suicide. In addition to the foregoing symptoms, a diagnosis of a major depressive episode require that the totality of symptoms presented must not meet the criteria for a mixed episode, and must cause clinically significant distress or impairment in social, occupational, or other important areas of functioning. Also, a major depressive episode is not diagnosed when the symptoms are due to the direct physiological effects of a substance (e.g., a drug of abuse, a medication), a general medical condition (e.g., hypothyroidism) or bereavement. Major clinical depression is diagnosed when a severely depressed mood persists for more than two weeks.

The extended release SAMe according to the present invention may be administered to a patient in need thereof: i.e. a patient who is either currently undergoing or is deemed to be in danger of undergoing a depressive episode, including a patient who has a history of depression or who is deemed to be at risk for depression. The pharmaceutically effective dose of SAMe administered to the patient may be in the range of about 400 mg/day to about 4000 mg/day, with common doses being about 400, 800, 1200, 1600, 2000 and 3200 mg/day. The effective dose of SAMe will relieve one or more symptoms of depression listed above, thereby partially or completely: lightening the patient's mood; restoring the patient's ability to experience pleasure; normalizing the patient's tendency to gain or loose weight; restoring the patient's normal sleep patterns; restoring the patient's normal psychomotor function; relieving the patient of fatigue; restoring the patient's feelings of self-worth; improving the patient's ability to concentrate and/or think clearly; or alleviating the patient's obsession with death. In particular, the extended release SAMe dosage form of the present invention is expected to lighten the patient's mood, restore the patient's ability to feel pleasure; and/or restore the patient's normal psychomotor function. In some specific embodiments of the invention, administration of the extended release SAMe formulation of the invention results in improvement in one or more symptoms of depression for a period starting within 1-4 weeks of administration It is contemplated that extended release S-adenosylmethionine may be characterized by 1. a more rapid onset of action; 2. higher adherence due to reduced frequency of dosing; 3. higher adherence due to reduced side-effects (see below); 4. higher percentage of patients gaining beneficial therapeutic effect due to 1, 2, and 3, as well as an independent effect of a more steady and sustained blood level of SAMe; and 5. reduced rate of induction of manic or other psychiatric or neurological symptoms due to 4. Thus it is contemplated the extended release formulation may decrease morbidity due to reasons 1, 2, 3, 4, and 5, and reduce the risk of suicidal behavior, suicide attempts or successful suicide due to reasons 1, 2, 3, 4, and 5. Consistent with above, it is contemplated that the steadier blood-level achieved by extended-release SAMe may be characterized by decreased side effects, especially side effects normally associated with high doses of SAMe, such as gastrointestinal effects (e.g. nausea, diarrhea, gas, constipation, anorexia (loss of appetite)) as well as head-ache, anxiety, insomnia, spasms, fatigue, hypomania and unmasking of mania.

Dysthymia

Dysthymia or dysthymic disorder is a form of depression characterized by a lack of enjoyment/pleasure in life that continues for at least two years. The symptoms of patients with dysthymic disorder are not as severe as those associated with major depressive disorder; however, the duration of these symptoms is much longer. While the symptoms of those suffering from dysthymia are less severe than those suffering clinical depression, over a lifetime dysthymia can have severe effects: high rates of suicide, work impairment, and social isolation. When a major depressive episode occurs on top of dysthymia, clinicians may refer to the resultant condition as double depression.

The Diagnostic and Statistical Manual of Mental Disorders (DSM), published by the American Psychiatric Association, characterizes Dysthymic Disorder as a chronic depression, but with less severity than a major depressive disorder. The essential symptom involves the individual feeling depressed almost daily for at least two years, but without the criteria necessary for a major depressive disorder. Low energy, disturbances in sleep or in appetite, and low self-esteem typically contribute to the clinical picture as well. Sufferers have often experienced dysthymia for many years before it is diagnosed. People around them come to believe that the sufferer is ‘just a moody person.’ The following diagnostic criteria are from the DSM-IV-TR, which is well-known to those of skill in the art: (1) On the majority of days for 2 years or more, the patient reports depressed mood or appears depressed to others for most of the day; (2) When depressed, the patient has 2 or more of: (a) appetite decreased or increased; (b) sleep decreased or increased; (c) fatigue or low energy; (d) poor self-image; (e) reduced concentration or indecisiveness; (f) feels hopeless; (3) During this 2 year period, the above symptoms are never absent longer than 2 consecutive months; (4) During the first 2 years of this syndrome, the patient has not had a Major Depressive Episode; (5) The patient has had no Manic, Hypomanic or Mixed Episodes; (6) The patient has never fulfilled criteria for Cyclothymic Disorder; (7) The disorder does not exist solely in the context of a chronic psychosis (such as Schizophrenia or Delusional Disorder); (8) The symptoms are not directly caused by a general medical condition or the use of substances, including prescription medications; (9) The symptoms cause clinically important distress or impair work, social or personal functioning.

As with other forms of depression, a number of treatments exist for dysthymia. Doctors most commonly use psychotherapy, including cognitive therapy, to help change the mind-set of the individual affected. Additionally doctors may prescribe a variety of antidepressant medications, with most individuals with dysthymia responding to fluoxetine and imipramine in a positive manner. For mild or moderate depression, the American Psychiatric Association in its 2000 Treatment Guidelines for Patients with Major Depressive Disorder advises psychotherapy alone or in combination with an antidepressant as possibly appropriate.

Because SAMe has proven effective in the treatment of other depressive disorders, such as depression (e.g. major clinical depression), it is expected that the extended release SAMe formulations of the invention will be effective in treating dysthymia. Contemplated dosages of extended release SAMe formulations for the treatment of dysthymia are from about 400 to about 3200 mg/day given on a once a day (or at most twice a day) basis.

Anxiety Disorders

The extended release SAMe formulations of the invention are contemplated for treatment of psychiatric disorders such as anxiety disorders. Among the anxiety disorders contemplated as being indicated for the extended release SAMe formulations of the present invention, there may be mentioned generalized anxiety disorder, post traumatic stress disorder, panic disorder or obsessive compulsive disorder. Because SAMe has proven effective in the treatment of other psychiatric disorders, such as depression, it is expected that the extended release SAMe formulations of the invention will be effective in treating anxiety disorders. Contemplated dosages of extended release SAMe formulations for the treatment of anxiety disorders are from about 400 to about 3200 mg/day given on a once a day (or at most twice a day) basis.

Generalized Anxiety Disorder

The frequency, intensity, and duration of the worry are disproportionate to the actual source of worry, and such worry often interferes with daily functioning. People with GAD often have a variety of symptoms such as tension, a tendency to be startled easily, restlessness, hyperactivity, worrying, fear, and excessive rumination. According to the DSM-UV, the symptoms must be consistent, persisting at least every other day and persisting for at least 6 months, in order to constitute GAD.

Patients who are generally nervous, depressed, unable to tolerate frustration and experience feelings of being inhibited are more likely to be diagnosed with GAD. People with GAD tend to have more conflicts with others and are very hard on themselves, they also tend to avoid common situations for fear of worry and. In youth GAD often leads too lower levels of social supports, academic underachievement, underemployment, substance use and high probability of obtaining other psychiatric. GAD differs from other anxiety disorders in the sense that there is no clear stimulus that elicits anxiety or appears to be the proximate cause of anxiety. It also lacks the clear avoidance and escape behaviors of phobias and, unlike panic attacks associated with most disorders, the anxiety levels associated with GAD are fairly moderate.

According to the Diagnostic and Statistical Manual IV-Text Revision (DSM-IV-TR), the following criteria must be met for a person to be diagnosed with Generalized Anxiety Disorder: (1) Excessive anxiety and worry (apprehensive expectation), occurring more days than not for at least six months, about a number of events or activities (such as work or school performance). (2) The person finds it difficult to control the worry. (3) The anxiety and worry are associated with three (or more) of the following six symptoms: (i) Restlessness or feeling keyed up or on edge; (ii) Being easily fatigued (difficulty concentrating or mind going blank); (iii) Irritability; (iv) Muscle tension; (v) sleep disturbance (difficulty falling or staying asleep, or restless unsatisfying sleep); (vi) Excessive sweating; (4) The focus of the anxiety and worry is not confined to features of an Axis I disorder, e.g., the anxiety or worry is not about having a panic attack (as in panic disorder), being embarrassed in public (as in social phobia), being contaminated (as in obsessive-compulsive disorder), being away from home or close relatives (as in Separation Anxiety Disorder), gaining weight (as in anorexia nervosa), having multiple physical complaints (as in somatization disorder), or having a serious illness (as in hypochondriasis), and the anxiety and worry do not occur exclusively during posttraumatic stress disorder; (5) The anxiety, worry, or physical symptoms cause clinically significant distress or impairment in social, occupational, or other important areas of functioning; (6) The disturbance is not due to the direct physiological effects of a substance (e.g., a drug of abuse, a medication) or a general medical condition (e.g., hyperthyroidism) and does not occur exclusively during a Mood Disorder, a Psychotic Disorder, or a Pervasive Developmental Disorder.

Because SAMe has proven effective in the treatment of other psychiatric disorders, such as depression, it is expected that the extended release SAMe formulations of the invention will be effective in treating generalized anxiety disorder. Contemplated dosages of extended release SAMe formulations for the treatment of generalized anxiety disorder are from about 400 to about 3200 mg/day given on a once a day (or at most twice a day) basis.

Post Traumatic Stress Disorder (PTSD)

Post-traumatic stress disorder (PTSD) is a term for certain psychological consequences of exposure to, or confrontation with, stressful experiences that the person experiences as highly traumatic. The experience must involve actual or threatened death, serious physical injury, or a threat to physical and/or psychological integrity. It is occasionally called post-traumatic stress reaction to emphasize that it is a routine result of traumatic experience rather than a manifestation of a pre-existing psychological weakness on the part of the patient.

Symptoms of PTSD can include the following: nightmares, flashbacks, emotional detachment or numbing of feelings (emotional self-mortification or dissociation), insomnia, avoidance of reminders and extreme distress when exposed to the reminders (“triggers”), irritability, hypervigilance, memory loss, and excessive startle response, clinical depression and anxiety, loss of appetite. The current diagnostic criteria for the PTSD published in the Diagnostic and Statistical Manual of Mental Disorders may be found DSM-IV-TR, and are thus known to those of skill in the art. Other symptoms can include general restlessness, insomnia, aggressiveness, depression, dissociation, emotional detachment and nightmares. A potential symptom is memory loss about an aspect of the traumatic event. Amplification of other underlying psychological conditions may also occur. Young children suffering from PTSD will often re-enact aspects of the trauma through their play and may often have nightmares that lack any recognizable content.

There are several known symptom clusters associated with PTSD: intrusion, hyperarousal, avoidance and dissociation. Intrusion arises from sufferers' inability to process the extreme emotions brought about by the trauma; they are plagued by recurrent nightmares or daytime flashbacks, during which they graphically re-experience the trauma. These re-experiences are characterized by high anxiety levels and make up one part of the PTSD symptom cluster triad called intrusive symptoms.

Hyperarousal refers to the characteristic state of nervousness experienced by PTSD sufferers, with the patient being prepared for “fight or flight”. The typical hyperactive startle reaction, characterized by “jumpiness” in connection with high sounds or fast motions, is typical for another part of the PTSD cluster called hyperarousal symptoms and could also be secondary to an incomplete processing.

Avoidance refers to the tendency of PTSD sufferers to avoid contact with everything and everyone, even their own thoughts, which may arouse memories of a traumatic event and thus provoke the intrusive and hyperarousal states. Sufferers isolate themselves, becoming detached in their feelings with a restricted range of emotional response and can experience so-called emotional detachment (“numbing”). This avoidance behavior is the third part of the symptom triad that makes up the PTSD criteria.

Dissociation is another psychological “defense” that includes a variety of symptoms, such as feelings of depersonalization and derealization, disconnection between memory and affect so that the person is “in another world,” and, in extreme forms can involve apparent multiple personalities and acting without any memory (“losing time”).

PTSD is commonly treated using a combination of psychotherapy (cognitive-behavioral therapy, group therapy, and exposure therapy are popular) and psychotropic drug therapy (antidepressant or atypical antipsychotics, e.g. fluoxetine, venlafaxin, sertraline, mirtazapine, olanzapine, or quetiapine. According to some studies, the most effective psychotherapeutic treatment for PTSD is Eye Movement Desensitization and Reprocessing (EMDR). Talk therapy may prove useful, but only insofar as the individual sufferer is enabled to come to terms with the trauma suffered and successfully integrate the experiences in a way that does not further damage the psyche. A technique of “rewriting” the content of nightmares through imagery rehearsal so that they have a resolution can not only reduce the nightmares but also other symptoms. The US Food and Drug Administration (FDA) recently approved a clinical protocol that combines the drug MDMA (“Ecstasy”) with talk therapy sessions.

PTSD is often co-morbid with other psychiatric disorders such as depression and substance abuse. Currently under scrutiny is the inclusion of Complex Post Traumatic Stress in the 2006 revision of the DSM-IV-TR. This is a variant of PTSD that includes the breakthrough of Borderline Personality traits.

Because SAMe has proven effective in the treatment of other psychiatric disorders, such as depression, and because PTSD possesses symptoms in common (and often co-morbid) with depression, it is expected that the extended release SAMe formulations of the invention will be effective in treating PTSD. Contemplated dosages of extended release SAMe formulations for the treatment of PTSD are from about 400 to about 3200 mg/day given on a once a day (or at most twice a day) basis.

Panic Disorder

Panic Disorder (PD; also known as cardiac neurosis or neurosis cordis) is a mental condition that causes the sufferer to experience sporadic panic attacks.

Panic disorder is characterized by a series of intense episodes of extreme anxiety, known as panic attacks. A panic attack may be triggered by an especially stressful situation, or it may occur for no particular reason. These events usually last for several minutes. Some individuals deal with these events on a regular basis—sometimes daily or weekly. Because of the constant fear of having another panic attack, individuals with panic disorder are often extremely uncomfortable in social situations, and may experience comorbid agoraphobia.

The DSM-IV provides the following criteria for diagnosing panic disorder: (1) recurrent unexpected panic attacks; (2) at least one of the attacks has been followed by 1 month (or more) of one (or more) of the following: (i) persistent concern about having additional attacks; (ii) worry about the implications of the attack or its consequences (e.g., losing control, having a heart attack, “going crazy”) (iii) a significant change in behavior related to the attacks; (3) Panic attack may be accompanied by agoraphobia; (4) The panic attacks are not due to the direct physiological effects of a substance (e.g., a drug of abuse, a medication) or a general medical condition (e.g., hyperthyroidism); (5) the panic attacks not better accounted for by another mental disorder, such as Social Phobia (e.g., occurring on exposure to feared social situations), Specific Phobia (e.g., on exposure to a specific phobic situation), Obsessive-Compulsive Disorder (e.g., on exposure to dirt in someone with an obsession about contamination), Posttraumatic Stress Disorder (e.g., in response to stimuli associated with a severe stressor), or Separation Anxiety Disorder (e.g., in response to being away from home or close relatives). It is considered that the skilled clinician will be familiar with PD and will be capable of diagnosing PD as appropriate.

Because SAMe has proven effective in the treatment of other psychiatric disorders, such as depression, and because PD shares common symptoms with depression, it is expected that the extended release SAMe formulations of the invention will be effective in treating PD. Contemplated dosages of extended release SAMe formulations for the treatment of PD are from about 400 to about 3200 mg/day given on a once a day (or at most twice a day) basis.

Obsessive Compulsive Disorder

Obsessive-compulsive disorder (OCD) is an anxiety disorder, characterized by a patient's obsessive, distressing, intrusive thoughts and related compulsions (tasks or rituals) which attempt to neutralize the obsessions.

According to the DSM-IV-TR, a diagnosis of OCD requires either or both of obsessions and compulsions. Obsessions are defined by: (1) recurrent and persistent thoughts, impulses, or images that are experienced at some time during the disturbance, as intrusive and inappropriate and that cause marked anxiety or distress; (2) the thoughts, impulses, or images are not simply excessive worries about real-life problems; (3) the person attempts to ignore or suppress such thoughts, impulses, or images, or to neutralize them with some other thought or action; (4) the person recognizes that the obsessional thoughts, impulses, or images are a product of his or her own mind.

Compulsions are defined by: (1) repetitive behaviors or mental acts that the person feels driven to perform in response to an obsession, or according to rules that must be applied rigidly; (2) the behaviors or mental acts are aimed at preventing or reducing distress or preventing some dreaded event or situation; however, these behaviors or mental acts either are not connected in a realistic way with what they are designed to neutralize or prevent or are clearly excessive; (3) in addition to these criteria, at some point during the course of the disorder, the sufferer must realize that his/her obsessions or compulsions are unreasonable or excessive; (4) The obsessions or compulsions must be time consuming (taking up more than one hour per day), cause distress, or cause impairment in social, occupational, or school functioning.

The DSM-IV-TR is well-known to those of skill in the art; and it is contemplated that the skilled clinician will be familiar with it or will consult it before diagnosing a patient with OCD. In many cases, OCD gives rise to feelings similar to those associated with depression.

The typical OCD sufferer performs tasks (or compulsions) to seek relief from obsession-related anxiety. To others, these tasks may appear odd and unnecessary. But for the sufferer, such tasks can feel critically important, and must be performed in particular ways to ward off dire consequences and to stop the stress from building up. Examples of these tasks: repeatedly checking that one's parked car has been locked before leaving it; turning lights on and off a set number of times before exiting a room; repeatedly washing hands at regular intervals throughout the day.

Obsessions are thoughts and ideas that the sufferer cannot stop thinking about. Common OCD obsessions include fears of acquiring disease, getting hurt, or causing harm to someone. Obsessions are typically automatic, frequent, distressing, and difficult to control or put an end to. People with OCD who obsess about hurting themselves or others are actually less likely to do so than the average person.

Compulsions refer to actions that the person willingly performs, most often repeatedly, in an attempt to cause the obsession to go away. For an OCD sufferer who obsesses about germs or contamination, for example, these compulsions often involve repeated cleansing or meticulous avoidance of trash and mess. Most of the time the actions become so regular that it is not a noticeable problem. Common compulsions include excessive washing and cleaning; checking; hoarding; repetitive actions such as touching, counting, arranging and ordering; and other ritualistic behaviors that the person feels will lessen the chances of provoking an obsession. Compulsions can be observable—washing, for instance—but they can also be mental rituals such as repeating words or phrases, or counting.

People who suffer from the separate condition obsessive compulsive personality disorder (OCPD) are not aware of anything abnormal about themselves; they will readily explain why their actions are rational, and it is usually impossible to convince them otherwise. People who suffer from OCPD tend to derive pleasure from their obsessions or compulsions, while those with OCD do not feel pleasure but are ridden with anxiety. OCD is ego dystonic, meaning that the disorder is incompatible with the sufferer's self-concept. Because disorders that are ego dystonic go against an individual's perception of his/herself, they tend to cause much distress. OCPD, on the other hand, is ego syntonic—marked by the individual's acceptance that the characteristics displayed as a result of this disorder are compatible with his/her self-image. Ego syntonic disorders understandably cause no distress (K. Carter, PSYC 210 lecture, Apr. 11, 2006). This is a significant difference between these disorders.

OCD is different from behaviors such as gambling addiction and overeating. People with these disorders typically experience at least some pleasure from their activity; OCD sufferers do not actively want to perform their compulsive tasks, and experience no pleasure from doing so.

OCD is placed in the anxiety class of mental illness, but like many chronic stress disorders it can lead to clinical depression over time. The constant stress of the condition can cause sufferers to develop a deadening of spirit, a numbing frustration, or sense of hopelessness. OCD's effects on day-to-day life—particularly its substantial consumption of time—can produce difficulties with work, finances and relationships.

OCD ranges widely in severity. There is no known cure for OCD, but it can be treated with anti-depressants.

Because SAMe has proven effective in the treatment of other psychiatric disorders, such as depression, and because OCD shares common symptoms with, and may give rise to, depression, it is expected that the extended release SAMe formulations of the invention will be effective in treating OCD. Contemplated dosages of extended release SAMe formulations for the treatment of OCD are from about 400 to about 3200 mg/day given on a once a day (or at most twice a day) basis.

Pain Disorders

There are a number of pain disorders of diverse (and in many cases unexplained) etiology, having a common element of pervasive or persistent pain. Such pain disorders include chronic lower back pain, chronic headaches, fibromyalgia, shingles, reflex sympathetic dystrophy and polyneuropathy. Chronic lower back pain may be mechanical, biochemical or psychogenic. Whatever its etiology, chronic lower back pain may in some instances be treated with an analgesic, such as aspirin, acetaminophen, hydrocodone or a combination of a non-NSAID drug and an opioid, such as hydrocodone or oxycodone.

Fibromyalgia

Fibromyalgia (FM or FMS) is a chronic syndrome characterized by diffuse or specific muscle, joint, or bone pain, fatigue, and a wide range of other symptoms. The primary symptom of fibromyalgia is widespread, diffuse pain, often including heightened sensitivity of the skin (allodynia), tingling of the skin (often needlelike), achiness in the muscle tissues, prolonged muscle spasms, weakness in the limbs, and nerve pain.

Chronic sleep disturbances are also characteristic of fibromyalgia, and some studies suggest that these sleep disturbances are the result of a sleep disorder called alpha wave interrupted sleep pattern, a condition in which deep sleep is frequently interrupted by bursts of brain activity similar to wakefulness. REM sleep is seldom reached during fibromyalgia-disturbed sleep. Many fibromyalgia patients experience “brain fog”, also known as “fibrofog”, exhibiting abnormally slow brain waves and cognitive deficits. Many experts suspect that “brain fog” is directly related to the sleep disturbances experienced by sufferers of fibromyalgia. It is not unusual for patients to experience extended periods (two hours or more) of ‘sleep inertia’.

Other symptoms often attributed to fibromyalgia (possibly due to another comorbid disorder) are chronic paresthesia, physical fatigue, irritable bowel syndrome, genitourinary symptoms such as those associated with the chronic bladder condition interstitial cystitis, dermatological disorders, headaches, myoclonic twitches, and symptomatic hypoglycemia. Although it is common for patients with fibromyalgia to experience widespread pain, fibromyalgia pain may also be localized in areas such as the shoulders, neck, back, hips, or other areas. Many sufferers also experience varying degrees of temporomandibular joint (TMJ) disorder. Not all patients have all symptoms.

Fibromyalgia can start as a result of some trauma (such as a traffic accident) or major surgery (usually hysterectomy), but there is currently no known strong correlation between any specific type of trigger and the subsequent initiation of fibromyalgia. Symptoms can have a slow onset, and many patients have mild symptoms beginning in childhood, such as growing pains. Symptoms are often aggravated by unrelated illness or changes in the weather. They can become more tolerable or less tolerable throughout daily or yearly cycles; however, many people with fibromyalgia find that, at least some of the time, the condition prevents them from performing normal activities such as driving a car or walking up stairs. The syndrome does not cause inflammation as is presented in arthritis, but anti-inflammatory treatments, such as ibuprofen and iontophoresis, are known to temporarily reduce pain symptoms in some people.

Some factors that have been associated with increased patient discomfort include: cold weather (especially when damp); changes in atmospheric pressure (such as with the onset of a cold front); malnutrition, hunger, or starvation; physical activity; lack of deep (REM) sleep; increase of stress. When making a diagnosis of fibromyalgia, a practitioner would take into consideration the patient's case history and the exclusion of other conditions such as endocrine disorders, arthritis, and polymyalgia rheumatica. There are also two criteria established by The American College of Rheumatology for diagnosis: a history of widespread pain lasting more than three months and tender points. There are 18 designated possible tender points (although a person with the syndrome may feel pain in other areas as well). During diagnosis, four kilograms-force (40 newtons) of force is exerted at each of the 18 points; the patient must feel pain at 11 or more of these points for fibromyalgia to be considered. Four kilograms of force is about the amount of pressure required to turn fingernails white or to feel pain sensations on the forehead. This technique was developed by the American College of Rheumatology as a means of confirming the diagnosis for clinical studies. It is also used in the United Kingdom. Pressure on nearby areas rarely elicits any reaction. Fibromyalgia patients also have elevated levels of Substance P in the body, which increases the levels of pain and intensity.

Because SAMe has proven effective in the treatment of other disorders, such as depression, and in particular other pain disorders, such as osteoarthritis, it is expected that the extended release SAMe formulations of the invention will be effective in treating fibromyalgia. Contemplated dosages of extended release SAMe formulations for the treatment of fibromyalgia are from about 400 to about 3200 mg/day given on a once a day (or at most twice a day) basis.

Other Pain Disorders

Shingles is a painful disease caused by the same virus, Herpes zoster, which causes chicken pox. The virus lays dormant after the patient has recovered from chicken pox, and then for some unexplained reason re-emerges after years of dormancy to infect and inflame the neurons branching from the spine. The patient experiences a rash and extreme pain, the latter of which may persist for days, weeks or months after the rash has resolved. Current treatment includes corticosteroid injections.

Chronic headaches, such as migraines and cluster headaches are a pervasive problem. Current treatments include strong analgesics, avoidance of so-called triggers (e.g. bright or flashing lights), and dietary adjustments.

Reflex sympathetic dystrophy (RSD) is characterized by scattered limb pain. The symptoms of RSD include: (1) pain—even with stimulus that are normally not painful; (2) weakness; (3) hypersensitivity; (4) skin changes; (5) swelling; (6) and sensations of cold Patients will often describe the pain as a burning or aching that ranges from mild discomfort to a feeling that is excruciating and intolerable.

RSD generally arises from some sort of trauma to a limb. Once a patient begins to experience RSD, the symptoms tend to continue long after the original injury. Often, the symptoms will even spread to areas of the limb not originally injured. RSD can become very debilitating.

Painful polyneuropathy is a relatively common syndrome, characterized by a painful numbness or burning in the hands or feet. In more severe cases, the pain spreads over time to the arms, legs or trunk, leading to muscle weakness. It is caused by damaged peripheral nerves. In contrast to nociceptive pain, which is caused by an injury to the body, neuropathic pain is the result of injury to the nerves themselves. Neuropathic pain occurs when damaged nerves misfire, signaling pain, even in the absence of a normally painful stimulus. When this type of pain becomes chronic, it is called neuropathic pain; and when it becomes widespread and chronic, it is referred to as polyneuropathy.

Painful polyneuropathy may arise from a number of different causative agents or events, including: diabetes, kidney failure, alcoholism, HIV infection/AIDS and chemotherapy. It is not known how neuropathy is caused by these causative agents or events. However, it is believed that neuropathic pain is characterized by damage to nerve fibers or to the myelin sheath that surrounds them.

Because SAMe has proven effective in the treatment of other disorders involving pain, such as osteoarthritis, and because pain disorders share common symptoms with osteoarthritis, it is expected that the extended release SAMe formulations of the invention will be effective in treating pain disorders. Contemplated dosages of extended release SAMe formulations for the treatment of pain disorders are from about 400 to about 3200 mg/day given on a once a day (or at most twice a day) basis.

### Liver Disease

A variety of liver disorders have been identified, which are expected to respond positively to extended release SAMe therapy, including alcoholic liver disease, fatty liver (also known as non-alcoholic fatty liver) and hepatitis. Hepatitis (inflammation of the liver) can be cause by a number of etiological agents, including viral and chemotoxic agents. SAMe has been tested extensively for efficacy in the treatment of liver disease; and it is expected that an extended release SAMe formulation according to the present invention will provide relief from one or more symptoms of liver disease.

Because SAMe has proven effective in the treatment of liver disorders, it is expected that the extended release SAMe formulations of the invention will also be effective in treating liver disorders. Contemplated dosages of extended release SAMe formulations for the treatment of liver disorders are from about 400 to about 3200 mg/day given on a once a day (or at most twice a day) basis.

Alcoholic Liver Disease

Alcohol abuse is a leading cause of morbidity and mortality throughout the world. It is estimated that in the United States as many as 10% of men and 3% of women may suffer from persistent problems related to the use of alcohol. The Fourth Edition of the Diagnostic and Statistical Manual of Mental Disorders (DSM-IV) published by the American Psychiatric Association divides alcohol use disorders into “alcohol dependence” and “alcohol abuse.” Alcohol dependence is indicated by evidence of tolerance and/or symptoms of withdrawal such as delirium tremens (DTs) or alcohol withdrawal seizures (rum fits) upon cessation of drinking. Alcohol affects many organ systems of the body, but perhaps most notably affected are the central nervous system and the liver. Almost all ingested alcohol is metabolized in the liver and excessive alcohol use can lead to acute and chronic liver disease. Liver cirrhosis resulting from alcohol abuse is one of the ten leading causes of death in the United States.

From data obtained in autopsy studies, it appears that between 10% and 15% of alcoholics have cirrhosis at the time of death. It is unknown why some alcoholics develop liver disease while others do not. One possibility is that there are genetic factors that predispose some alcoholics to liver disease. Some data also suggest that co-factors such as chronic infection with hepatitis C virus may increase the risk of the development of cirrhosis in an alcoholic. In general, women who drink an equal amount of alcohol are at higher risk than men for the development of liver disease, possibly because of decreased metabolism of alcohol in the stomach prior to absorption.

Fatty Liver

Fatty liver (or steatorrhoeic hepatosis or steatosis hepatis) is a reversible condition where large vacuoles of lipid accumulate in hepatocytes (the cells of the liver). It may be caused by various diseases, such as in chronic alcoholism and obesity. Accumulation of triglycerides (fat) in liver cells may cause the liver to enlarge.

Many chemicals, such as alcohol and drugs can cause fatty liver. Fatty liver can also occur in diabetes mellitus and in pregnancy (acute fatty liver of pregnancy). It can also be seen both in starvation (especially rapid weight loss) and in obesity. In addition, it is also a minor symptom of hepatitis that may indicate progression to cirrhosis.

Fatty change represents the intracytoplasmic accumulation of triglyceride (neutral fats). At the beginning, the hepatocytes present small fat vacuoles (liposomes) around the nucleus—microvesicular fatty change. In the late stages, the size of the vacuoles increases pushing the nucleus to the periphery of the cell—macrovesicular fatty change. These vesicles are well delineated and optically empty because fats dissolves during tissue processing. Large vacuoles may coalesce, producing fatty cysts—which are irreversible lesions.

Severe fatty liver is accompanied by inflammation, a situation that is referred to as steatohepatitis. The degree of inflammation is related to its progression to more severe forms of liver disease, ultimately cirrhosis. If this occurs in a non-alcoholic patient without viral liver disease, the condition is termed non-alcoholic steatohepatitis.

Because SAMe has proven effective in the treatment of liver disorders, it is expected that the extended release SAMe formulations of the invention will be effective in treating liver disorders such as fatty liver. Contemplated dosages of extended release SAMe formulations for the treatment of fatty liver are from about 400 to about 3200 mg/day given on a once a day (or at most twice a day) basis.

Hepatitis

Hepatitis is a gastroenterological disease, featuring inflammation of the liver. The clinical signs and prognosis, as well as the therapy, depend on the cause. Hepatitis is an inflammation of the liver characterized by malaise, joint aches, abdominal pain, vomiting 2-3 times per day for the first 5 days, loss of appetite, dark urine, fever, hepatomegaly (enlarged liver) and jaundice (icterus, yellowing of the eyes and skin). Some chronic forms of hepatitis show very few of these signs and are only present when the longstanding inflammation has led to the replacement of liver cells by connective tissue; this disease process is referred to as cirrhosis of the liver. Certain liver function tests can also indicate hepatitis.

Viral Hepatitis: Most cases of acute hepatitis are due to viral infections: hepatitis A; hepatitis B; hepatitis; hepatitis B with D; hepatitis E; hepatitis G. In addition to the hepatitis viruses, some other viruses can cause hepatitis, including cytomegalovirus, Epstein-Barr virus, yellow fever, etc.

Hepatitis A: Hepatitis A or infectious jaundice is an Hepatovirus (originally thought to be an enterovirus) transmitted by the orofecal route, transmitted to humans through methods such as contaminated food. It causes an acute form of hepatitis and does not have a chronic stage. The patient's immune system makes antibodies against hepatitis A that confer immunity against future infection. People with hepatitis A are advised to rest, stay hydrated and avoid alcohol. A vaccine is available that will prevent infection from hepatitis A for life. Hepatitis A can be spread through personal contact, consumption of raw sea food or drinking contaminated water. This occurs primarily in third world countries. Strict personal hygiene and the avoidance of raw and unpeeled foods can help prevent an infection. Infected persons already begin excreting the hepatitis A virus with their stool two weeks after the appearance of the first symptoms. The time between the infection and the start of the illness can run from 15 to 45 days, and approximately 15% of sufferers may experience relapsing symptoms from six months to a year following initial diagnosis.

Hepatitis B: Hepatitis B can cause both acute and chronic hepatitis. Chronic hepatitis develops in the 15% of patients who are unable to eliminate the virus after an initial infection. Identified methods of transmission include blood (blood transfusion, now rare), tattoos (both amateur and professionally done), sexually (through sexual intercourse or through contact with blood or bodily fluids), or in utero (from mother to her unborn child, as the virus can cross the placenta). However, in about half of cases the source of infection cannot be determined. Blood contact can occur by sharing syringes in intravenous drug use, shaving accessories such as razor blades, or touching wounds on infected persons. Needle-exchange programs have been created in many countries as a form of prevention. In the United States, 95% of patients clear their infection and develop antibodies against hepatitis B virus. About 5% of patients do not clear the infection and develop chronic infection; only these people are at risk of long term complications of hepatitis B. Patients with chronic hepatitis B have antibodies against hepatitis B, but these antibodies are not enough to clear the infection that establishes itself in the DNA of the affected liver cells. The continued production of virus combined with antibodies is a likely cause of immune complex disease seen in these patients. A vaccine is available that will prevent infection from hepatitis B for life. Hepatitis B infections result in 500,000 to 1,200,000 deaths per year worldwide due to the complications of chronic hepatitis, cirrhosis, and hepatocellular carcinoma. Hepatitis B is endemic in a number of (mainly South-East Asian) countries, making cirrhosis and hepatocellular carcinoma big killers. There are three FDA-approved treatment options available for persons with a chronic hepatitis B infection: alpha-interferon, adefovir and lamivudine. In about 45% of persons on treatment achieve a sustained response.

Hepatitis C: Hepatitis C (originally “non-A non-B hepatitis”) can be transmitted through contact with blood (including through sexual contact where the two parts blood is mixed). Hepatitis C may lead to a chronic form of hepatitis, culminating in cirrhosis. It can remain asymptomatic for 10-20 years. No vaccine is available for hepatitis C. Patients with hepatitis C are prone to severe hepatitis if they contract either hepatitis A or B, so all hepatitis C patients should be immunized against hepatitis A and hepatitis B if they are not already immune. However, hepatitis C itself is a very lethal virus and can cause cirrhosis of the liver. The virus, if detected early on can be treated by a combination of interferon and the antiviral drug ribavirin. The genotype of the virus determines the rate of response to this treatment regimen.

Hepatitis E: Hepatitis E produces symptoms similar to hepatitis A, although it can take a fulminant course in some patients, particularly pregnant women; it is more prevalent in the Indian subcontinent.

Hepatitis G: Another type of hepatitis, hepatitis G, has been identified, and is probably spread by blood and sexual contact. There is, however, doubt about whether it causes hepatitis, or is just associated with hepatitis, as it does not appear to be primarily replicated in the liver.

Drug induced hepatitis: A large number of drugs can cause hepatitis. The anti-diabetic drug troglitazone was withdrawn in 2000 for causing hepatitis. However, many patients lack one or more of the cytochrome P-450 enzymes needed to metabolize many chemicals, including pharmaceuticals. Drug-induced hepatitis may arise in a seemingly healthy patient who has been exposed to an agent that is not generally toxic to the liver because the patient is unable to metabolize the agent, which then accumulates at hepatotoxic levels in the liver.

Because SAMe has proven effective in the treatment of liver disorders, it is expected that the extended release SAMe formulations of the invention will also be effective in treating hepatitis, especially hepatitis A, B, C or drug-induced (chemotoxic) hepatitis. Contemplated dosages of extended release SAMe formulations for the treatment of hepatitis are from about 400 to about 3200 mg/day given on a once a day (or at most twice a day) basis.

### Extended Release Same Formulations

The present invention provides extended release SAMe compositions for twice a day (b.i.d.) or in some preferred embodiments once a day (q.d.) administration. A variety of methods have been used to prepare extended release compositions of various drugs; and it is contemplated that at least one of these methodologies can be used to prepare extended release SAMe compositions according to the present invention. For example, U.S. Pat. No. 6,759,395 (incorporated herein in its entirety) provides gelatin capsules capable of being adapted to provide extended release of SAMe, e.g. by including within the gelatin capsules granules of SAMe coated with a controlled-release coating, optionally including a pore former, such as sodium alginate and/or a fatty acid, such as stearic acid, or another water-soluble pore former. The types of extended release SAMe compositions that are contemplated within the scope of the present invention include osmotic dosage forms, extended release matrices, pulsatile release formulations and extended release formulations coated with one or more enteric coatings. In some embodiments, an extended release matrix (monolithic core) containing SAMe may be coated with an extended release coating, which may optionally include a pore former (such as sodium alginate, stearic acid or both). Thus, an ER formulation of SAMe according to the invention will include any formulation that has, as a substantial part of that formulation, an extended release component comprising SAMe—that is a component that releases SAMe over a period of more than about 2 hours, particularly about 2 to 24, 3 to 24 or 4 to 24 hours. As SAMe is sensitive to oxidation, in some embodiments it is considered necessary to coat the SAMe with a coating that will protect the SAMe from oxidation. The coating may be applied directly to SAMe granules (e.g. by spraying an oxygen impermeable coating, which may be an enteric coating, an immediate release coating, an extended release coating or a combination thereof, onto SAMe granules in a fluidized bed) or may be applied to the outside of a tablet, capsule or other dosage form, e.g. by spraying or dipping a tablet or capsule core containing SAMe. In some embodiments, the dosage form is a tablet or caplet containing SAMe in a matrix or osmotic core and the oxygen impermeable layer is applied by spraying the oxygen impermeable layer onto the outside of the matrix or osmotic core or by dipping the matrix or osmotic core into a solution containing the oxygen impermeable layer material. In some embodiments, the oxygen impermeable layer is an enteric coating. In some embodiments, the oxygen impermeable layer, e.g. an immediate release layer, is applied before an enteric coating is applied to the outside of the dosage form, either by spraying the enteric coating onto the dosage form or dipping the core into the coating material. It is contemplated that a method of coating that results in an oxygen impermeable layer being interposed between the SAMe and the outside of the dosage form will produce a suitable result.

Granulation of SAMe: In some embodiments, SAMe is granulated before incorporating it into the dosage form. Granulation may be used to form particulates of suitable size and consistency for further processing, which may include coating of the particulates, compaction of the particulate into tablets, combination of the particulates with one or more excipients, including matrix formers, diluents, glidants, lubricants, anti-caking materials, etc.

In some embodiments, the granulation method is a wet-granulation method. In some embodiments, for example, a water soluble salt of SAMe is dissolved in a suitable solvent, such as water, and is sprayed into a drying environment, e.g. a heated stream of dry air. Other embodiments are also possible. In some embodiments, granulation of SAMe may also be accomplished by one or more dry granulation methods. In some such embodiments, the dry granulation method is a slugging method. Slugging is a dry granulation method in which SAMe, optionally in combination with one or more excipients, is first compressed to form a slug and is then milled to form particulates suitable for further processing. In some embodiments, the granulation method is roller compaction method, in which powder size enlargement is accomplished by feeding SAMe, optionally in combination with one or more wet or dry excipients (e.g. binders), through a roller apparatus, followed by drying (if necessary), milling and sizing the compacted SAMe mixture to form granules having the desired size.

In some embodiments, the granulated SAMe may then be coated by spraying the SAMe with a coating material, such as an oxygen-impermeable coating material, an enteric coating material, a coating that retards release of SAMe from the granule, or a combination of two or more thereof, and then incorporated into a suitable dosage form. For example, granulated SAMe may be spray coated first with an oxygen-impermeable layer and then an enteric coating and introduced into a gelatin capsule of appropriate size or may be further combined with one or more excipients (e.g. one or more binders, matrix formers, diluents, anti-caking agents, etc.) and compacted into tablets, caplets or cores for osmotic extended release formulations. As another example, granulated SAMe may be spray coated with an extended release layer and introduced into a gelatin capsule of appropriate size or may be further combined with one or more excipients (e.g. one or more binders, matrix formers, diluents, anti-caking agents, etc.) and compacted into tablets, caplets or cores for osmotic extended release formulations. In other embodiments, granulated SAMe may be spray coated with an oxygen-impermeable layer, then incorporated into an extended release matrix, which, after compaction to form a tablet or caplet, may then be coated with an enteric coating, an immediate release coating, a slow-release coating or some combination of two or more thereof. In other embodiments, the granulated SAMe may be incorporated into an extended release matrix to form a core, which is then coated with a coating, such as an immediate release coating that also serves as an oxygen-impermeable layer. The coated core then may be coated with an enteric coating, or in some embodiments, may be used as-is. In other embodiments, the granulated SAMe may be incorporated into an extended release matrix to form a core, which may then be coated with an enteric coating that is also oxygen-impermeable.

In some embodiments, the granules of SAMe obtained from wet- or dry-compaction methods, may be divided into two populations, one of which receives a first coating and a second of which receives a second coating having different properties from the first coating. The different properties of the coatings are due to differences in chemical properties, physical properties or both. In terms of chemical properties, the coatings may differ in terms of composition (e.g. one coating could be an extended release coating having a first composition and the second could be an extended release coating having a different composition), in terms of physical properties (e.g. one coating can be thicker than the other) or both. In terms of physical properties, the mass of a coating in relation to the final mass of the population of granules (“relative mass”) may be easily calculated and a difference in coating thickness between two populations of particles may be inferred where the populations have substantially the same particle size distribution and the two coatings have substantially the same composition. In some embodiments, the first and second coatings are different in terms of their thickness and/or relative weights. In some embodiments, the first and second coatings have the same composition, but differ in terms of their thickness and/or relative weights. In some embodiments, the first and second coatings are both of the same or similar thickness and/or relative weights, but differ in composition. In some embodiments, the first and second coatings are both delayed release coatings (which optionally may also be oxygen-impervious), but differ in thickness and/or relative weights. In some embodiments, the two populations of granules may then be introduced into a capsule (e.g. a gel capsule) or may be compacted into a tablet or caplet. In some specific embodiments, the first population of granules is coated with a first thickness of an extended-release or controlled-release coating and the second population of granules is coated with a second thickness of the same or different extended-release or controlled-release coating; then the two populations of granules are combined with one or more excipients, such as binders, diluents, anti-caking agents, etc., and then compacted to form tablets, tablet cores or caplets. Tablet cores may be further coated, for example with an enteric coating, an osmotic coating (which may also contain pore-formers and/or a laser-drilled hole), an anti-oxidant coating, a protective coating or other coating. The proportion of the first population of granules to the second population of granules in the single dosage form (tablet, core, caplet, capsule, etc.) may be adjusted to achieve a desired release profile. In some embodiments, the proportion of the first population of granules to the second population of granules is in the range or 1:20 to 20:1 (by SAMe weight). In some embodiments, the two populations of granules may be combined with a third, coated or uncoated, population of granules. The coating on the third population of granules, if present, will be different from those of the first and second populations of granules. In some such embodiments, the ratios of first and second, second and third and first and third populations of granules will be 1:20 to 20:1, 1:20 to 20:1 and 1:20 to 20:1 (by SAMe weight), respectively.

In some embodiments, the granules of SAMe obtained from wet- or dry-compaction methods, may be divided into two populations, one of which is further coated and the other of which is not, before the two populations of granules are combined in a single dosage form. The coated population receives a coating and is then combined in a capsule (e.g. a gel capsule) or may be compacted into a tablet, tablet core or caplet. In forming a tablet, core or caplet, the two populations of granules are optionally combined with one or more excipients, such as binders, diluents, anti-caking agents, etc.; then the granules, optionally admixed with excipients, are compacted to form tablets, tablet cores or caplets. Tablet cores may be further coated, for example with an enteric coating, an osmotic coating (which may also contain pore-formers and/or a laser-drilled hole), an anti-oxidant coating, a protective coating and/or other coating. The proportion of the first population of granules to the second population of granules in the single dosage form (tablet, core, caplet, capsule, etc.) may be adjusted to achieve a desired release profile. In some embodiments, the proportion of the first population of granules to the second population of granules is in the range or 1:20 to 20:1 (by SAMe weight).

Extended Release Matrices: Matrix tablet systems incorporating active ingredients, fillers, binders and various other types of excipients have been employed with various active pharmaceutical ingredients (APIs) to provide extended release dosage forms. For example, hydroxypropyl cellulose (HPMC) has been used together with other matrix constituents, such as ethylcellulose, methylcellulose, sodium carboxymethyl cellulose, etc., to form controlled release delivery systems. (See: U.S. Pat. No. 4,601,894; U.S. Pat. No. 4,687,757; and U.S. Pat. No. 4,695,591, each incorporated herein by reference.) Hydroxypropyl cellulose and a carboxy vinyl polymer have also been used. (See U.S. Pat. No. 4,680,323, incorporated herein by reference). A hydrophilic matrix comprising a free-flowing directly compressible granulation useful as a controlled release pharmaceutical excipients a heteropolysaccharide and a polysaccharide material capable of cross-linking the heteropolysaccharide. (See U.S. Pat. No. 4,994,276, incorporated herein by reference.) Indeed, various extended release matrices have been prepared using one or more alkylated cellulose derivatives, such as methyl cellulose, ethyl cellulose, hydroxymethyl cellulose, hydroxypropyl cellulose, hydroxypropyl methyl cellulose, etc. (See: U.S. Pat. No. 4,389,393; U.S. Pat. No. 4,525,345; U.S. Pat. No. 4,556,678; U.S. Pat. No. 4,692,337; U.S. Pat. No. 4,756,911; U.S. Pat. No. 5,073,380; U.S. Pat. No. 4,968,509; U.S. Pat. No. 5,462,747; U.S. Pat. No. 5,543,154; U.S. Pat. No. 5,439,687; U.S. Pat. No. 5,264,446, each of which is incorporated herein by reference in its entirety.) In some embodiments, SAMe is combined with a matrix former and optionally one or more hydrophobic barrier forming agents and/or one or more anti-caking agents (e.g. micronized silicon dioxide and/or magnesium aluminum silicate). In some embodiments, SAMe is combined with magnesium aluminometasilicate and optionally one or both of light liquid paraffin and/or magnesium stearate, subjected to granulation (e.g. slugging or roller compaction, as described herein), combined with one or more excipients (e.g. one or more anti-caking agents) and then compacted to form tablets or tablet cores. In some specific embodiments, SAMe is combined with appropriate amounts of magnesium aluminometasilicate, light liquid paraffin and magnesium stearate; then the mixture is slugged and combined with additional magnesium stearate; finally the mixture is compacted to form tablets or tablet cores.

Osmotic Formulations

Osmotic type extended release tablets are externally similar in appearance to conventional tablets. However, the interior of the osmotic formulation includes an osmotically active drug core surrounded by a semipermeable membrane. The core is divided into two layers: an “active” layer containing the drug, and a “push” layer containing pharmacologically inert (but osmotically active) components. The membrane surrounding the tablet is permeable to water but not to drug or osmotic excipients. As water from the gastrointestinal is imbibed into the tablet, pressure increases in the osmotic layer and “pushes” against the drug layer, resulting in the release of drug through a small, laser-drilled orifice in the membrane on the drug side of the tablet. Drug delivery is essentially constant as long as the osmotic gradient remains constant, and then gradually falls to zero. The biologically inert components of the tablet remain intact during GI transit and are eliminated in the feces as an insoluble shell.

Osmotic formulations comprising two layers and coated with an extended release coating having an aperture therein have been used to provide zero-order release. In general, the formulations are prepared by preparing a first, osmotic layer, which is overlayed with a second, matrix layer comprising the API and at least one matrix component. The two layers are then coated with a semi-permeable coating. (The semi-permeable coating is permeable to water, but not the API). The first semi permeable coating may be coated with a second semi-permeable coating, in which case the inner semi-permeable coating may incorporate a pore forming component, which is gradually dissolved, thereby permitting increased rate of water ingress over time. An aperture is then formed through the water-permeable coating or coatings, which permits egress of the API under osmotic influence of the water imbibed through the water-permeable coating or coatings. (See for example US 2005/0158382.)

Other osmotic release compositions are formed by mixing an API with an insoluble swelling agent and forming an osmotic core, about which is press formed a semi-permeable coating having an aperture therein. (See U.S. Pat. No. 6,365,185, incorporated herein by reference.)

Enteric Coating: Due to the relative instability of SAMe in gastric fluids (pH˜1-4), in some embodiments it may be necessary to coat the extended release SAMe compositions of the present invention with an enteric coating. In general, the enteric coating may be any pharmaceutically acceptable coating that is insoluble in the stomach (pH˜1-4), but is soluble at the prevailing pH of the intestines (pH˜6-8). The enteric coating should also be inert with respect to the portion of the tablet that it coats. In this regard, it is considered possible to coat the extended release core with an intermediate coating, such as an immediate release coating, and then to coat the intermediate coating with an enteric coating. Thus the intermediate coating (e.g. the immediate release coating) can, in some embodiments of the contemplated invention, provide an inert barrier between the enteric coating and the extended release core. This type of structure may be used, whether the extended release core is of the matrix type or the osmotic core type. Indeed, US 2005/0158382 describes both osmotic and matrix-type extended release cores which may be spray or dip coated with either an enteric coating that does not react with the extended release core, or with an immediate release coating that is coated with an enteric coating.

Pulsatile Release

In some embodiments, the formulation of the present invention may comprise a controlled-release pharmaceutical composition comprising SAMe that is capable of delivering therapeutic amounts of SAMe to the proximal small bowel, distal small bowel or colonic regions of the gastrointestinal tract of an animal. In some embodiments, the present invention provides a controlled-release pharmaceutical composition comprising SAMe which may comprise the following components, each of which includes SAMe: (A) an immediate-release (IR) component of SAMe which is released within about 1 hour after administration; and (B) a delayed-release (DR) component comprising of SAMe which is released in the body over a period of time of about 2 hours to about 24 hours, about 3 to about 24 hours or about 4 to about 24 hours after administration. In some embodiments, the invention contemplates a multiparticulate controlled-release composition having a first component comprising a first population of SAMe-containing particles and a second component comprising a second population of SAMe-containing particles. The first component may be an immediate-release component, a controlled-release component or a delayed-release component having a first release profile. The active ingredient-containing particles of the second component may be coated with a controlled-release coating or may be provided in a controlled-release matrix material. In embodiments in which the SAMe-containing particles of the second component are coated with a controlled-release coating, the coating applied to the second population of particles causes a delay between the release of SAMe from the first population of particles and the release of SAMe from the second population of particles. Similarly, the presence of a controlled-release matrix material in the second population of particles causes a delay between the release of SAMe from the first population of particles and the release of SAMe from the second population of particles. The duration of the delay may be varied by altering the composition and/or the amount of the controlled-release coating and/or altering the composition and/or amount of controlled-release matrix material utilized. Thus, the duration of the delay can be designed to achieve a desired plasma profile. Following oral delivery, the composition in operation is capable of delivering the active ingredient or active ingredients in a pulsatile manner.

As discussed in more detail above, multiparticulate compositions may comprise two or more populations of granules. A first population of granules may be coated with a first coating and a second population of granules may be coated with a second coating or may lack any coating. In any case, the first population and the second population differ from one another in terms of the physical, chemical or physico-chemical properties of their respective coatings. In some embodiments, dissolution of a first population of granules may be delayed by a first delay period by coating the granules with a delayed-release or controlled-release coating, while dissolution of the second population of granules may be delayed by a lesser delay period (including no delay) by coating the second population of granules with a faster-dissolving coating, a thinner layer of coating or no coating. In some embodiments, a dissolution profile of a dosage comprising more than one population of granules at pH 6-8 will demonstrate a multimodal dissolution profile over time. In some embodiments, a dissolution profile of a dosage comprising more than one population of granules at pH 1-4 (e.g. pH 1) will demonstrate a multimodal dissolution profile over time. In some embodiments, a blood plasma concentration curve for SAMe obtained after administration of a dosage comprising more than one population of granules will be multimodal over time. In some embodiments, a blood plasma concentration curve for SAMe obtained after administration of a dosage comprising more than one population of granules will demonstrate a blood plasma concentration curve for SAMe that is essentially flat—i.e. it varies less than about 10%, 15%, 30% or 40% (above baseline) over a period of at least about 6, 8, 10 or 12 hours.

The multiparticulate controlled-release composition of the invention may further comprise one or more additional active ingredients that are compatible with SAMe and, if more than one additional active ingredient, each other. In some embodiments, the multiparticulate controlled-release composition of the invention may comprise a therapeutically effective amount of the controlled-release form of SAMe of the present invention in combination with B12, folate or both. In some exemplary embodiments, the SAMe particulates may be coated separately from particulates containing B12 and/or folate in order to prevent interaction between SAMe and/or folate. The B12 and/or folate may be incorporated into an immediate-release or controlled-release formulation, e.g. by coating particulates containing B12 and/or folate with an appropriate immediate-release or controlled-release coating.

Because the plasma profile produced by the multiparticulate controlled-release formulation of the invention upon administration is substantially similar to the plasma profile produced by the administration of two or more immediate-release dosage forms given sequentially, the multiparticulate controlled release composition of the present invention is particularly useful for administering active ingredients for which such plasma profiles are desired. It is contemplated that the controlled-release composition will support q.d. dosing, although in some embodiments, b.i.d. dosing is also contemplated.

The present invention also provides solid oral dosage forms of SAMe comprising a composition according to the invention. The solid oral dosage forms of the present invention may further comprise B12, folate or both.

The time release characteristics for the release of the SAMe from each of the components may be varied by modifying the composition of each component, including modifying any of the excipients or coatings which may be present. In particular, the release of SAMe may be controlled by changing the composition and/or the amount of the controlled-release coating on the particles, if such a coating is present. If more than one controlled-release component is present, the controlled-release coating for each of these components may be the same or different. Similarly, when controlled-release is facilitated by the inclusion of a controlled-release matrix material, release of the SAMe may be controlled by the choice and amount of controlled-release matrix material utilized. The controlled-release coating may be present, in each component, in any amount that is sufficient to yield the desired delay time for each particular component. The controlled-release coating may be preset, in each component, in any amount that is sufficient to yield the desired time lag between components.

The delay for the release of the SAMe from each component may also be varied by modifying the composition of each of the components, including modifying any excipients and coatings which may be present. For example, the first component may be an immediate-release component from which the SAMe is released substantially immediately upon entry into the small intestine. The second component may be, for example, an extended-release component in which the SAMe is released in a controlled fashion over an extended period of time.

As will be appreciated by those skilled in the art, the exact nature of the plasma concentration curve will be influenced by the combination of all of the aforementioned factors. In particular, the delay between the delivery (and thus also the onset of action) of the SAMe in each component may be controlled by varying the composition and coating, if present, of each of the components. Thus, by variation of the composition of each component and by variation of the delay, numerous release and plasma profiles may be obtained. Depending on the duration of the delay between the release of SAMe from each component and the nature of the release from each component (i.e. immediate release, sustained release etc.), the pulses in the plasma profile may be well separated and clearly defined peaks (e.g. when the delay is long) or the pulses may be superimposed to a degree (e.g. in when the delay is short).

In another embodiment, the multiparticulate controlled-release composition according to the present invention has an immediate-release component and at least one controlled-release component, the immediate-release component comprising a first population of SAMe-containing particles and the controlled-release components comprising second and subsequent populations of SAMe-containing particles. The second and subsequent controlled-release components may comprise a controlled-release coating. Additionally or alternatively, the second and subsequent controlled-release components may comprise a controlled-release matrix material. In operation, administration of such a multiparticulate controlled-release composition having, for example, a single controlled-release component results in characteristic pulsatile plasma concentration levels of the SAMe in which the immediate-release component of the composition gives rise to a first peak in the plasma profile and the controlled-release component gives rise to a second peak in the plasma profile. Embodiments of the invention comprising more than one controlled-release component give rise to further peaks in the plasma profile.

A multiparticulate controlled-release composition according to the present invention may be incorporated into any suitable dosage form that facilitates release of the active ingredient in a pulsatile manner. For example, the dosage form may be a blend of the different populations of active ingredient containing particles which make up the immediate-release and the controlled-release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multilayer tablet, in which the first component of the multiparticulate controlled-release composition may be compressed into one layer, with the second component being subsequently added as a second layer of the multilayer tablet.

Preferably, in operation, the composition of the invention and the solid oral dosage forms containing the composition release the active ingredient such that substantially all of the active ingredient contained in the first component is released prior to release of the active ingredient from the second component. When the first component may comprise an immediate release component, for example, it is preferable that release of the active ingredient from the second component is delayed until substantially all the active ingredient in the immediate release component has been released. Release of the active ingredient from the second component may be delayed as detailed above by the use of a controlled-release coating and/or a modified release matrix material.

Dosing with Multiple Dosage Units

In some embodiments, the present invention provides for treatment of one or more diseases selected from the group consisting of osteoarthritis, rheumatoid arthritis fibromyalgia, a psychiatric disorder, an inflammatory condition, a central nervous system (CNS) disorder, a pain disorder and a liver disorder in a patient, comprising administering to the patient an extended release dosage comprising a therapeutically effective amount of S-adenosyl methionine (SAMe), or a pharmaceutically acceptable salt thereof. In some particular embodiments, the therapeutically effective dose is administered on a once-a-day basis. In some embodiments, the once-a-day dose may be administered in a single dosage unit—e.g. a single tablet, capsule, caplet, etc. In other embodiments, the dose may be administered as multiple tablets, capsules or caplets. In some embodiments, for instance, a dosage of 400 to 3200 mg of SAMe per day may be divided into two, three, four or more tablets, capsules or caplets of 100 to 1600 mg of SAMe per unit dose. In some preferred embodiments, the daily dose is two, three or four tablets, capsules or caplets of 100 to 800 mg of SAMe per dose. Particular dosage regimens that may be mentioned are: four units of 200, 400 or 800 mg SAMe per unit; three units of 100, 150, 200, 300, 400, 600, 800 or 1,000 mg of SAMe per unit; two units of 200, 400, 800 or 1600 mg per unit. In each case, the form of the dosage unit may be a capsule, a tablet, a caplet or other suitable extended release dosage unit.

In some embodiments, the extended release SAMe may be divided between multiple daily doses. In some particular embodiments, the extended release SAMe may be divided into two daily doses. Each dose may be administered as a single dosage unit—e.g. a single tablet, capsule or caplet—or may be divided into multiple dosage units. In some embodiments, a twice-daily dose of from about 100 to about 1600 mg of SAMe per dose may be divided into one to four dosage units of from about 100 to about 800 mg of SAMe per unit. In each case, the form of the dosage unit may be a capsule, a tablet, a caplet or other suitable extended release dosage unit.

Fed vs. Fasted Dosing

In some embodiments of the invention, it may be advantageous to ensure that the patient is either fed or fasted (e.g. overnight for at least about 6, especially about 8, hours). It is considered that food or a carbonated beverage administered at the same time, immediately (i.e. less than about 30, especially less than about 15 minutes) before or soon (e.g. less than about 10 minutes) after the extended release SAMe formulation of the invention is administered to the patient may increase the rate of gastric emptying, thus increasing the rate of uptake of SAMe from the extended release formulation. Thus, in some embodiments, the invention contemplates administering the extended release SAMe formulation of the invention with food or a carbonated beverage. In such cases, it is considered that the onset of action of SAMe will be hastened without significantly affecting the long-acting characteristics of the extended release SAMe formulation.

Combinations of Same with Other Active Ingredients

In some embodiments of the invention SAMe may be combined with one or more active ingredients, such as folate, B12, a compound for the treatment of one or more of the following: osteoarthritis, rheumatoid arthritis fibromyalgia, a psychiatric disorder, an inflammatory condition, a central nervous system (CNS) disorder, a pain disorder and a liver disorder. As suppressed levels of folate, B12 or both are correlated with lowered SAMe production, it is considered that combining SAMe with folate, B12 or both may result in increased supplementation of SAMe by enhancing the body's natural ability to make SAMe while at the same time supplementing SAMe with exogenous extended release SAMe. In some embodiments, SAMe may be combined with another active ingredient, such as folate, B12 or both, or other active ingredient, in a single dosage form. In other embodiments, SAMe may be administered separately from the active ingredient, such as folate, B12 or both. In some such embodiments, the extended release SAMe dosage form according to the invention may be included in a kit with a separate dosage form containing another active ingredient, such as folate, B12 or both, one or more compounds for the treatment of one or more of the following: osteoarthritis, rheumatoid arthritis fibromyalgia, a psychiatric disorder, an inflammatory condition, a central nervous system (CNS) disorder, a pain disorder and a liver disorder. In some embodiments of such a kit, the kit also includes instructions for co-administering SAMe and the one or more additional ingredients.

Combination Therapy with SAMe and Folate

In some embodiments of the invention, SAMe may be combined with folate (vitamin B9) in a single dosage form. The single dosage form may also contain one or more additional active ingredients, such as B12, as described below. The reference daily intake (RDI) for folate is in the range of about 400 μg/day for healthy males, about 600 μg/day for pregnant females and about 500 μg/day for lactating females. It is considered that supplementation of up to 1000 μg/day of folic acid may be co-administered with the extended release SAMe of the present invention. In this regard, it is noted that the folate may be admixed with the extended release SAMe according to the invention, or may be contained in an immediate release coating on the outside of the extended release SAMe dosage form, or may be contained in an immediate release core on the inside of the extended release SAMe dosage form.

In some embodiments of the invention, SAMe and folate may be administered in separate dosage forms. Each of the separate dosage forms may contain one or more additional active ingredients, such as B12, as described below. It is considered that the folate dosage form may contain from about 1 to about 1000 μg, specifically about 200 to about 1000 μg, more specifically about 400 to about 600 μg of folate per day. In some such embodiments, the extended release SAMe dosage form and the folate dosage form may be packaged in a kit for co-administration of the two dosage forms. In some specific embodiments, the kit may also include instructions for co-administration of the two dosage forms. The two dosage forms may be administered simultaneously or at different times of the day.

Combination Therapy with SAMe and B12

In some embodiments of the invention, SAMe may be combined with B12 in a single dosage form. The single dosage form may also contain one or more additional active ingredients, such as folate, as described above. The minimum recommended daily requirement for B12 ranges from about 1 μg per day in Europe to about 2.4 μg per day in the United States, with ranges of 0.1 to about 10 μg per day being suggested for supplementation to correct B12 deficiency. B12 is common in foods, such as meat, poultry, eggs and cheese. A non-vegetarian diet may contain as much as 1 to 2 mg of B12 per day. While the upper limits of the tolerated dose of B12 have not yet been determined, it is considered that the from about 1 to about 2000 μg, specifically about 2 to about 1000 μg, more specifically about 4 to about 100 μg of B12 per day would be a suitable dose for co-administration of B12 with the extended release SAMe according to the invention. In this regard, it is noted that the B12 may be admixed with the extended release SAMe according to the invention, or may be contained in an immediate release coating on the outside of the extended release SAMe dosage form, or may be contained in an immediate release core on the inside of the extended release SAMe dosage form.

In some embodiments of the invention, SAMe and B12 may be administered in separate dosage forms. Each of the separate dosage forms may contain one or more additional active ingredients, such as folate, as described above. It is considered that the B12 dosage form may contain from about 1 to about 2000 μg, specifically about 2 to about 1000 μg, more specifically about 4 to about 100 μg of B12 per day. In some such embodiments, the extended release SAMe dosage form and the B12 dosage form may be packaged in a kit for co-administration of the two dosage forms. In some specific embodiments, the kit may also include instructions for co-administration of the two dosage forms. In some embodiments, the two forms may be administered simultaneously or at different times of the day.

In some embodiments, SAMe may be administered as two or more dosage forms. In some embodiments, the dosage forms may be immediate release and extended release formats. In some embodiments, the immediate release format may be enteric coated. In some embodiments, the dosage forms may be two separate extended release forms. In some embodiments, the dosage forms may be two of the same monolithic core or capsule coated with two different coatings or the same coatings of different thicknesses (or relative weights with respect to the total dosage). In some embodiments, the SAMe may be administered as a first set of 1, 2, 3, 4 or 5 units (e.g. tablets or caplets) of an immediate release or 0%-4% (extended or controlled release coating) coated extended release core and a second set of 1, 2, 3, 4 or 5 units (e.g. tablets or caplets) that are different from the first set and have 3%-6% coated with an extended or controlled release coating. Such extended or controlled release coating may also contain about 5-50% or about 10-40% pore former.

### EXAMPLES

### Example 1

### Extended Release Monolithic Matrix Tablets

A formulation comprising SAMe, magnesium aluminometasilicate, light liquid paraffin and magnesium stearate was compounded by mixing the ingredients and compressing them with a semi-automatic tablet press. Humidity was maintained at less than 30% and temperature was maintained at 20-25° C. during the entire manufacturing process. The proportions of the ingredients are set forth in Table 1-1, below.

TABLE 1-1
Formulation of SAMe with Liquid Paraffin

| Excipients | Mg/Tablet | % (wt.) |
|---|---|---|
| SAMe | 400 | 72.7% |
| Magnesium Aluminometasilicate (Neusilin US 2) | 100 | 18.18 |
| Light Liquid Paraffin | 30 | 5.45 |
| Magnesium Stearate NF | 20 | 3.63 |
| Total wt of uncoated tablet (mg) | 500 | |

The formulation in Table 1-1 enabled manufacture of SAMe tablets with less than 30% total excipients. The granules used this formulation had good flow properties and demonstrated no sticking picking during compression.

### Example 2

### Slugging Procedure

In an effort to improve the compressibility of the SAMe formulation from Example 1, a granulation procedure (slugging) was employed. SAMe was mixed with liquid paraffin and magnesium aluminometasilicate. The resulting powder mixture was loaded into a V blender and mixed for 10 minutes at 50 RPM. Half the quantity of magnesium stearate (see Table 2-1, below), 2.97 g, was added to the V blender and mixed for another 10 minutes.

The resulting powder was passed through a 20 # sieve. The blend was compressed into 400-500 mg slugs with a hardness of about 8-9 kp. The slugs were then milled, passed through a 30 # sieve and mixed with the remaining magnesium stearate (2.97 g). The resulting mixture was then compressed to a hardness of 12-15 kp.

TABLE 2-1
Formulation for Manufacturing SAMe Tablet Core with Liquid Paraffin

| | | | Excipient Mass |
|---|---|---|---|
| Excipients | Mg/Tablet | % (wt) | for 110 Tablets |
| SAMe | 800 | 71.81 | 88.00 |
| Magnesium aluminometasilicate | 200 | 17.95 | 22.00 |
| Liquid Paraffin | 6.00 | 5.39 | 66.00 |
| Magnesium Stearate | 5.40 | 4.85 | 59.40 |
| Total | 114.00 | 100.00 | 122.54 |

Ethylcellulose coating was performed by third party vendors (Colorcon, Aqualon). The specific ethylcellulose coatings and their coating levels (percent weight gains upon coating) are shown in Table 2-2, below. Dissolution studies were performed in pH 6.8 PBS media using USP II dissolution apparatus and protocol.

TABLE 2-2
Ethylcellulose Based Coatings

| Coating Material | Coating Level (% Weight Gain on Coating) |
|---|---|
| Surelease ® by Colorcon | 5, 6, 7, 8 and 9% |
| Aquarius ® by Aqualon | 4, 5, 6 and 7% |

Introduction of the slugging process increased density of the powder and improved flow properties. Both coating trials were successful with no reported tablet erosion, delamination or friability during coating.

### Example 3

### Coating Trials

Matrix core SAMe tablets as disclosed in Example 2, above, were coated with ethylcellulose coatings having various amounts of pore former (Nutrateric® pore former, a combination of sodium alginate and purified stearic acid). The ethylcellulose portion of the coating was a combination of purified water, Ethyocel 20 cP STD. Prem. ethylcellulose and 28% ammonium hydroxide. The coatings tested were 100:0 (ethylcellulose:pore former), 80:20 and 70:30 by weight. Tablets were either uncoated or coated with either 2.5% of 70:30 or 80:20 ethylcellulose composition. Dissolution was tested in pH 6.8 PBS buffer solution. The results are summarized in Table 3-1:

TABLE 3-1
Dissolution Results for Uncoated
and Coated Tablets at pH 6.8

| | | Tablets Coated with | Tablet Coated with |
|---|---|---|---|
| | Uncoated | Ethylcellulose | Ethylcellulose |
| Time (hr) | Core | 70:30*, 2.5%** | 80:20*, 2.0%** |
| 2 | 56.36 | 22.72 | 10.17 |
| 3 | 64.00 | 28.72 | 15.99 |
| 4 | 74.21 | 33.78 | 22.73 |
| 6 | 78.27 | 41.92 | 34.09 |
| 8 | 82.00 | 49.19 | 43.22 |
| 10 | 87.53 | 53.11 | 49.95 |
| 12 | 88.22 | 57.32 | 54.68 |
| 15 | 86.96 | 62.29 | 61.15 |
| 18 | 84.08 | 65.26 | 66.48 |

*Ratio of ethylcellulose to pore former;
**Wt. % gain of Coating per Tablet

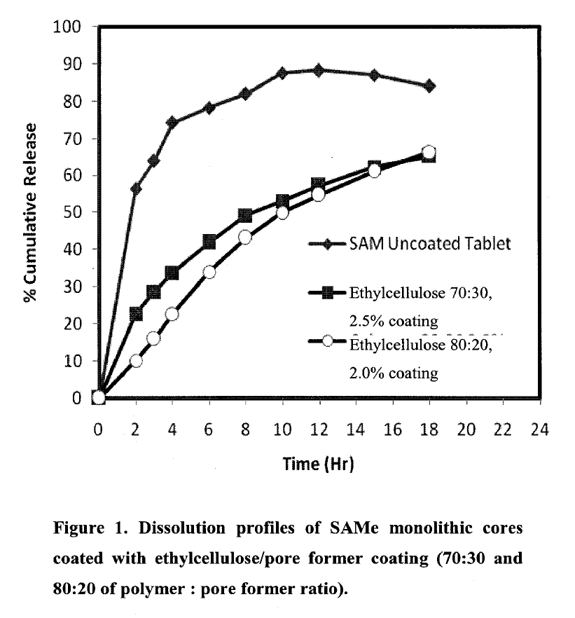

The results of this study are depicted graphically in FIG. 1.

### Example 4

### Second Coating Trial

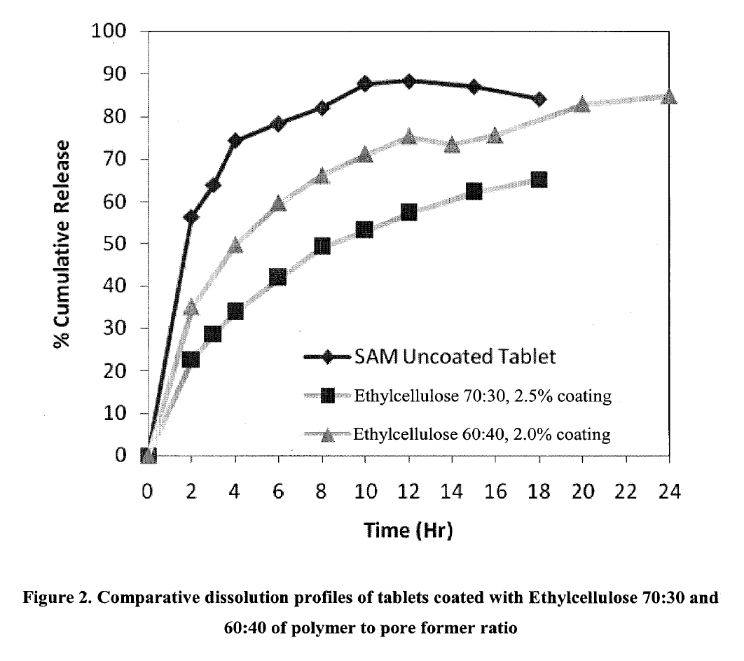

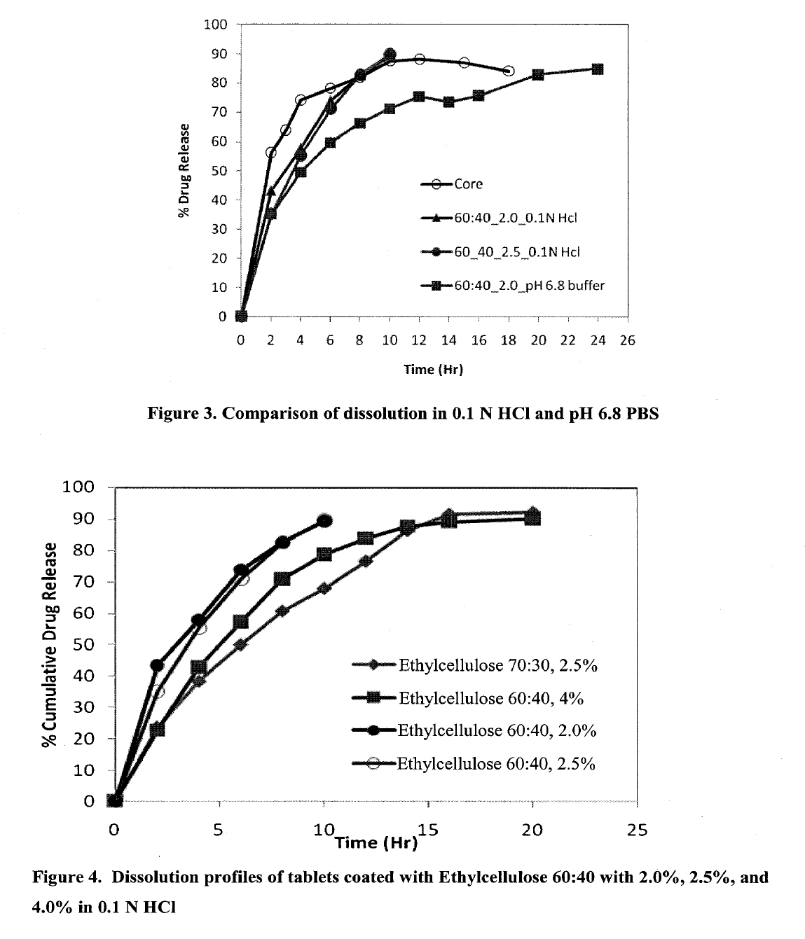

Tablets cores as described in Example 2 were coated with ethylcellulose coatings at a polymer to pore former ratio of 60:40 at various coating levels. The coating levels, as determined by weight gain, were 2.0, 2.5, 3.0, 3.5 and 4.0% weight gain. Dissolution studies were performed in pH 6.8 (starting pH) PBS and 0.1 N HCl using USP II dissolution apparatus. The results of this study are summarized in Table 4-1 and in FIGS. 2, 3 and 4. In Table 4-1, the ratio of polymer (ethylcellulose) to pore former is expressed as a ratio (e.g. 60:40, 70:30) and the coating level is expressed as wt. % weight gain over the uncoated core (e.g. 2.0%, 2.5%, 4%).

TABLE 4-1
Dissolution Results of Coated Tablets

| | Tablet coated | Tablet coated | Tablet coated | Tablet coated | Tablet coated |
|---|---|---|---|---|---|
| Time (hr) | with | with | with | with | with |
| Dissolution | 60:40, 2.0% | 60:40, 2.0% | 60:40, 2.5% | 60:40, 4% | 70:30, 2.5% |
| Medium | pH 6.8 PBS | 0.1 N HCl | 0.1 N HCl | 0.1 N HCl | 0.1 N HCl |
| 2 | 35.16 | 43.43 | 35.18 | 22.7 | 23.8 |
| 4 | 49.68 | 57.90 | 55.39 | 43.0 | 38.4 |
| 6 | 59.67 | 73.97 | 71.13 | 57.4 | 50.1 |
| 8 | 66.25 | 82.67 | 82.67 | 71.1 | 60.9 |
| 10 | 71.15 | 89.47 | 89.83 | 78.9 | 68.0 |
| 12 | 75.32 | | | 84.1 | 76.7 |
| 14 | 73.45 | | | 87.9 | 86.4 |
| 16 | 75.67 | | | 89.3 | 91.7 |
| 20 | 82.89 | | | 90.3 | 92.3 |

In pH 6.8 buffer, 70-75% of SAMe was released from the tablet coated with 2.0% of 60:40 polymer:pore former composition. It is considered that degradation of SAMe in the pH 6.8 solution may have led to degradation of the drug during the study, reducing the concentration of SAMe in the course of the study at pH 6.8. In order to test this hypothesis, parallel studies were conducted in pH 1 (0.1 N HCl) solution. Both 60:40, 4% and 70:30, 2.5% coatings provided dissolution profiles in pH 1 solution that were considered to meet extended-release criteria. Such compositions are considered suitable for advancement into in vivo studies in man or animal models.

### Example 5

### Human (in Vivo) Administration of Extended-Release Coated Matrix Cores

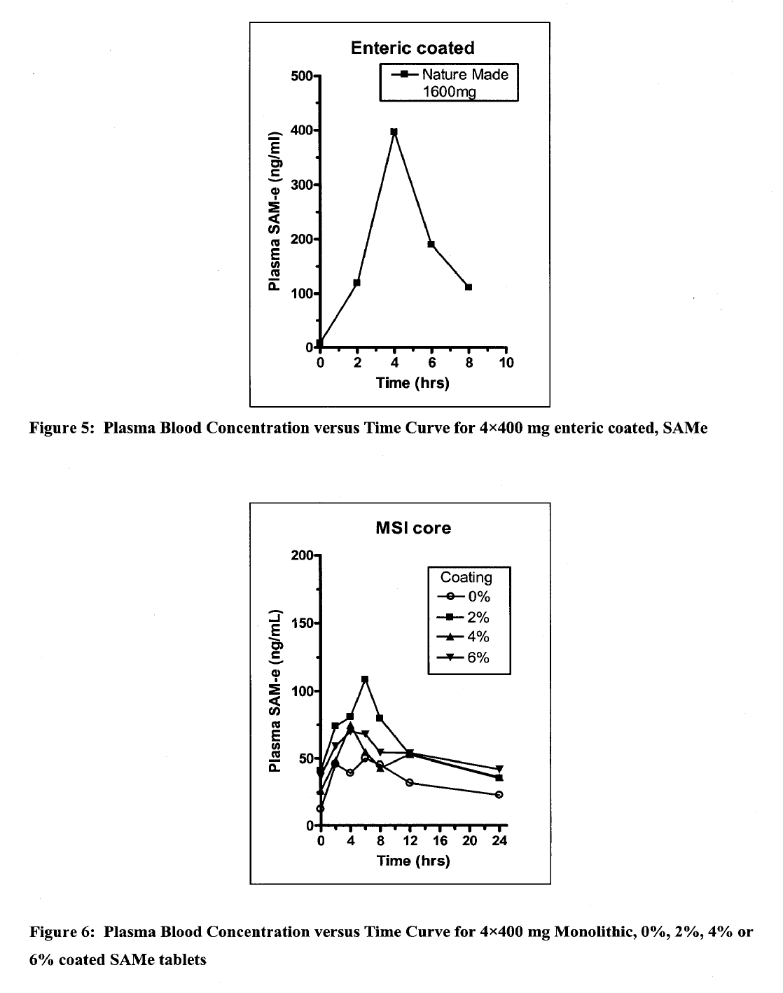

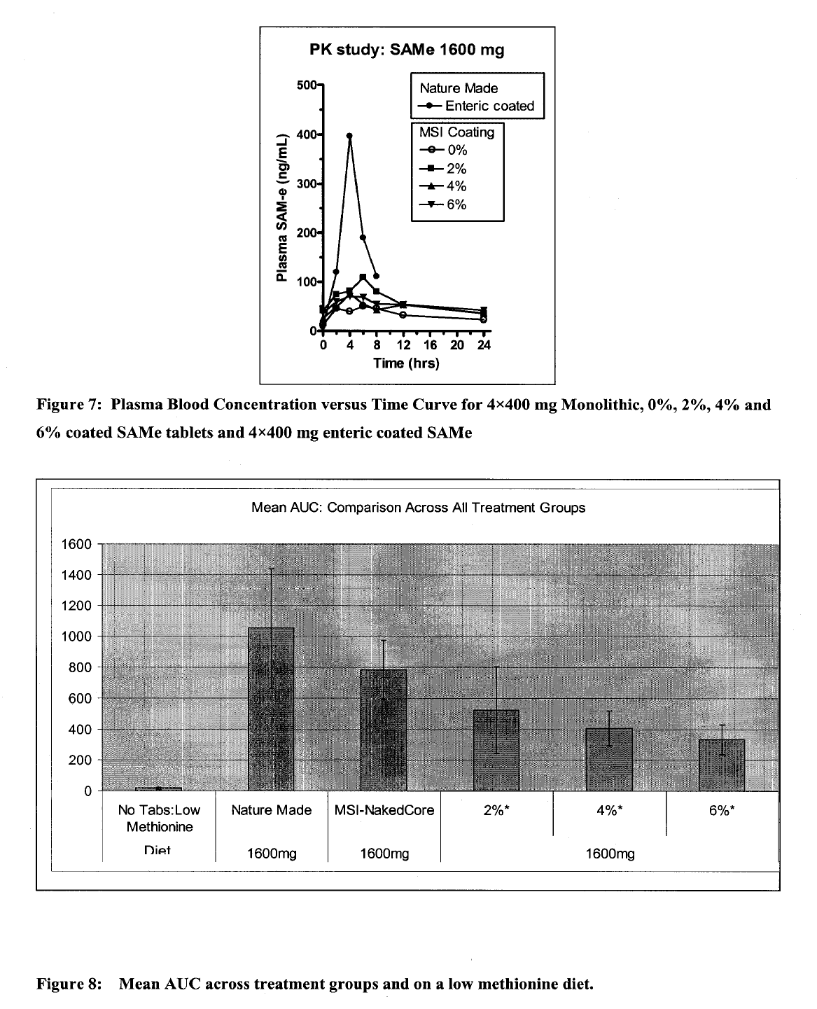

In order to understand the in vivo release characteristics of coated and uncoated monolithic SAMe tablets, the SAMe cores having the composition set forth in Example 2 were coated with 0%, 2%, 4% or 6% ethylcellulose (60:40 polymer to pore former ratio) and administered to human volunteers in an unblinded, pharmacokinetic study. The results obtained with the monolithic cores were compared to those obtained with commercially available SAMe in an enterically coated formulation (Mood Plus®, 4×400 mg enterically coated, immediate release SAMe Nature Made®). Blood samples were collected immediately before administration of SAMe (to establish baseline values) and at the intervals stated in Tables 5-1 through 5-7, below. The results of the study are depicted graphically in FIGS. 5, 6 and 7.

TABLE 5-1
SAMe Monolithic Core, 0% Coating: 640 mg of SAMe Ion

| | | | | | | | | Above | | |
|---|---|---|---|---|---|---|---|---|---|---|
| Time | | | | | Mean | | | Baseline | | |
| (hrs) | N-1 | N-2 | N-3 | N-4 | (ng/mL) | SD | N | (ng/mL) | C/Cmax | nmol/L |
| 0 | 18.3 | 3.5 | 3.7 | 23.5 | 12.3 | 10.2 | 4 | 0 | 0.00 | 30.74 |
| 2 | 17.4 | 3.7 | 27.0 | 135.0 | 45.8 | 60.3 | 4 | 33.5 | 0.89 | 114.85 |
| 4 | 54.2 | 14.6 | 16.1 | 71.8 | 39.2 | 28.5 | 4 | 26.9 | 0.71 | 98.26 |
| 6 | 98.3 | 10.5 | 8.0 | 83.2 | 50.0 | 47.5 | 4 | 37.7 | 1.00 | 125.46 |
| 8 | 127.4 | 5.8 | 23.4 | 24.9 | 45.4 | 55.4 | 4 | 33.1 | 0.88 | 113.92 |
| 12 | 42.4 | 12.4 | 41.8 | 30.6 | 31.8 | 14.1 | 4 | 19.6 | 0.52 | 79.81 |
| 24 | 40.0 | 6.7 | 23.9 | 21.1 | 22.9 | 13.6 | 4 | 10.7 | 0.28 | 57.55 |

C = [SAME]_{T} − [SAME]₀
Cmax = [SAME]ₘₐₓ − [SAME]₀

TABLE 5-2
SAMe Monolithic Core, 0% Coating: 1600 mg of SAMe Ion

| Time (hrs) | Z-1 | Z-2 | Z-3 | Z-4 | Z-5 |
|---|---|---|---|---|---|
| 0 | 19.5 | 18.0 | 12.4 | 19.4 | 20.5 |
| 2 | | | | | 196.2 |
| 4 | 133.2 | 105.2 | 53.5 | 59.4 | 139.5 |
| 8 | 56.3 | 74.0 | 29.9 | 53.0 | 37.4 |
| 12 | 31.8 | 66.7 | 23.9 | 28.9 | 43.9 |
| 24 | 23.4 | 35.8 | 17.6 | 28.5 | 22.3 |
| 32 | 24.4 | 57.4 | 21.0 | 21.2 | 27.7 |
| 48 | 23.9 | 24.8 | 16.7 | 16.4 | 27.0 |

TABLE 5-3
SAMe Monolithic Core, 2% Coating: 1600 mg of SAMe Ion

| | | | | | | | Above | | |
|---|---|---|---|---|---|---|---|---|---|
| | | | | Mean | | | Base- | | |
| Time | | | | (ng/ | | | line | C/ | |
| (hrs) | M2-1 | M2-2 | M2-3 | mL) | SD | N | (ng/mL) | Cmax | nmol/L |
| 0 | 20.4 | 48.0 | 54.8 | 41.1 | 18.2 | 3 | 0 | 0.00 | 103.07 |
| 2 | 57.8 | 108.6 | 56.2 | 74.2 | 29.8 | 3 | 33.1 | 0.49 | 186.26 |
| 4 | 80.0 | 85.8 | 77.6 | 81.1 | 4.3 | 3 | 40.1 | 0.59 | 203.59 |
| 6 | 75.0 | 207.4 | 43.9 | 108.8 | 86.8 | 3 | 67.7 | 1.00 | 272.96 |
| 8 | 60.7 | 116.1 | 62.3 | 79.7 | 31.5 | 3 | 38.6 | 0.57 | 200.05 |
| 12 | 42.4 | 84.8 | 31.2 | 52.8 | 28.3 | 3 | 11.8 | 0.17 | 132.58 |
| 24 | 32.4 | 48.7 | 24.8 | 35.3 | 12.2 | 3 | −5.8 | −0.09 | 88.61 |

C = [SAME]_{T} − [SAME]₀
Cmax = [SAME]ₘₐₓ − [SAME]₀

TABLE 5-4
SAMe Monolithic Core, 4% Coating, 1600 mg of SAMe Ion

| | | | | | | | Above | | |
|---|---|---|---|---|---|---|---|---|---|
| | | | | Mean | | | Base- | | |
| Time | | | | (ng/ | | | line | C/ | |
| (hrs) | M4-1 | M4-2 | M4-3 | mL) | SD | N | (ng/mL) | Cmax | nmol/L |
| 0 | 23.1 | 24.6 | 30.6 | 26.1 | 4.0 | 3 | 0 | 0.00 | 65.49 |
| 2 | 45.5 | 41.3 | 58.6 | 48.5 | 9.1 | 3 | 22.4 | 0.46 | 121.64 |
| 4 | 42.8 | 36.8 | 145.0 | 74.9 | 60.8 | 3 | 48.8 | 1.00 | 187.93 |
| 6 | 20.6 | 34.1 | 109.7 | 54.8 | 48.0 | 3 | 28.7 | 0.59 | 137.57 |
| 8 | 29.8 | 32.7 | 66.0 | 42.9 | 20.1 | 3 | 16.8 | 0.34 | 107.55 |
| 12 | 59.0 | 51.0 | 49.6 | 53.2 | 5.1 | 3 | 27.1 | 0.56 | 133.52 |
| 24 | 27.0 | 37.0 | 43.9 | 36.0 | 8.5 | 3 | 9.9 | 0.20 | 90.25 |

C = [SAME]_{T} − [SAME]₀
Cmax = [SAME]ₘₐₓ − [SAME]₀

TABLE 5-5
SAMe Monolithic Core, 6% Coating, 1600 mg of SAMe Ion

| | | | | | | | | Above | | |
|---|---|---|---|---|---|---|---|---|---|---|
| Time | | | | | Mean | | | Baseline | | |
| (hrs) | M6-1 | M6-2 | M6-3 | M6-4 | (ng/mL) | SD | N | (ng/mL) | C/Cmax | nmol/L |
| 0 | 38.0 | 32.4 | 40.5 | 36.2 | 36.8 | 3.4 | 4 | 0 | 0.00 | 92.34 |
| 2 | 61.9 | 41.2 | 42.2 | 92.4 | 59.4 | 24.0 | 4 | 22.6 | 0.68 | 149.16 |
| 4 | 98.3 | 65.4 | 49.0 | 67.2 | 70.0 | 20.6 | 4 | 33.2 | 1.00 | 175.58 |
| 6 | 65.0 | 91.6 | 56.8 | 59.0 | 68.1 | 16.0 | 4 | 31.3 | 0.94 | 170.89 |
| 8 | 65.2 | 50.4 | 58.3 | 44.2 | 54.5 | 9.2 | 4 | 17.7 | 0.53 | 136.83 |
| 12 | 81.8 | 33.3 | 47.3 | 53.6 | 54.0 | 20.4 | 4 | 17.2 | 0.52 | 135.55 |
| 24 | 47.7 | 37.0 | 42.6 | 40.6 | 42.0 | 4.5 | 4 | 5.2 | 0.16 | 105.34 |

C = [SAME]_{T} − [SAME]₀
Cmax = [SAME]ₘₐₓ − [SAME]₀

TABLE 5-6
Enteric Coated Monolithic (ER) Core, 1600 mg of SAMe Ion

| | EM-1 | | EM-2 | | EM-3 | | Mean | Mean | |
|---|---|---|---|---|---|---|---|---|---|
| Time | μmol/ | | μmol/ | | μmol/ | | μmol/ | ng/ | C/Cmax |
| (hrs) | L | ng/ml | L | ng/ml | L | ng/ml | L | ml | μmol/L |
| 0 | 50.1 | 20.0 | 95.3 | 38.1 | 100.4 | 40.2 | 81.9 | 32.6 | 0.00 |
| 2 | 74.2 | 29.7 | 136.7 | 54.7 | 133.1 | 53.2 | 114.7 | 45.7 | 0.82 |
| 4 | 90.9 | 36.4 | 124.6 | 49.8 | 142.4 | 57.0 | 119.3 | 47.5 | 0.94 |
| 6 | 68.9 | 27.6 | 142.4 | 57.0 | 154.3 | 61.7 | 121.9 | 48.6 | 1.00 |
| 8 | 115.2 | 46.1 | 165.3 | 66.1 | 141.3 | 56.5 | 140.6 | 56.0 | 1.47 |
| 12 | 70.4 | 28.2 | 159.8 | 63.9 | 128.3 | 51.3 | 119.5 | 47.6 | 0.94 |
| 24 | 66.4 | 26.6 | 139.8 | 55.9 | 156.5 | 62.6 | 120.9 | 48.2 | 0.98 |

C = [SAME]_{T} − [SAME]₀
Cmax = [SAME]ₘₐₓ − [SAME]₀

TABLE 5-7
Enteric Coated Immediate Release (Nature Made ®) Core, 1600 mg of SAMe Ion

| Time | NM-1 | | NM-2 | | NM-3 | | NM-4 | | Mean | Mean | |
|---|---|---|---|---|---|---|---|---|---|---|---|
| (hrs) | μmol/L | ng/ml | μmol/L | ng/ml | μmol/L | ng/ml | μmol/L | ng/ml | μmol/L | ng/ml | C/Cmax |
| 0 | 34.9 | 14.0 | 155.5 | 62.2 | 32.3 | 12.9 | 119.4 | 47.8 | 85.5 | 34.1 | 0.00 |
| 2 | 37.4 | 15.0 | 253.0 | 101.2 | 35.8 | 14.3 | 150.0 | 60.0 | 119.1 | 47.4 | 0.56 |
| 4 | 1427 | 570.9 | 501.2 | 200.5 | 28.0 | 11.2 | 149.5 | 59.8 | 526.5 | 209.8 | 0.64 |
| 6 | 939.4 | 375.8 | 577.7 | 231.1 | 1001.4 | 400.6 | 151.1 | 60.4 | 667.4 | 265.9 | 0.68 |
| 8 | 289.7 | 115.9 | 221.6 | 88.6 | 268.4 | 107.4 | 117.1 | 46.8 | 224.2 | 89.3 | 1.00 |
| 12 | 88.9 | 35.6 | 124.1 | 49.6 | 124.9 | 50.0 | 96.1 | 38.4 | 108.5 | 43.2 | 0.64 |
| 24 | 32.5 | 13.0 | 161.2 | 64.5 | 47.7 | 19.1 | 83.1 | 33.2 | 81.1 | 32.3 | 0.66 |

C = [SAME]_{T} − [SAME]₀
Cmax = [SAME]ₘₐₓ − [SAME]₀

As can be seen from FIGS. 5 through 7, the monolithic core in accordance with Example 2, provided extended increase in blood plasma concentrations of SAMe above baseline, whereas the enteric coated formulation provided a rapid rise in SAMe concentration in blood plasma, followed by precipitous decline. The blood concentration profiles set forth in Tables 5-1 through 5-4 are very flat, demonstrating little change between hours 2 and 4, hours 4 and 6, hours 6 and 8 and hours 8 and 12, whereas the enteric coated SAMe formulation showed a nearly 300% variance between hours 2 and 4, and a nearly 200% variance between hours 4 and 6. It is considered that the flat blood plasma concentration curve obtained in Tables 5-1 through 5-4 are desirable from the standpoint of providing a more even release of SAMe over time.

Using the data provided above, the area under the plasma concentration (AUC) values were calculated for the Immediate Release (Nature Made®), Extended Release (Monolithic) core, and the 60:40-coated Extended Release core (2%, 4% and 6%). The values are set forth in the following Table 5-7.

TABLE 5-7
AUC Values for Immediate Release and 0%, 2%, 4% and 6% Coated
Monolithic Core

| | Baseline | | 1600 mg | | | |
|---|---|---|---|---|---|---|
| | No | | MSI- | 1600 | 1600 | 1600 |
| | Tabs:Low | 1600 mg | NakedCore | mg | mg | mg |
| | Methionine | Nature Made | (0%) | 2%* | 4%* | 6%* |
| Average | 16.0 | 1052 | 782.1 | 526.1 | 407.5 | 334.2 |
| AUC | | | | | | |
| sem | 5.3 | 388 | 191.0 | 280.0 | 112.9 | 97.2 |

The data shown above are depicted graphically in FIG. 8.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.95 when T is about 2 hours; Q is about 0.5 to about 1.0 when T is about 4 hours; Q is about 0.5 to about 1.0 when T is about 6 hours; Q is about 0.3 to about 0.9 when T is about 8 hours; and Q is about 0.15 to about 0.6 when T is about 12 hours.

2. The method of claim 1, wherein the disorder is a liver disorder selected from the group consisting of alcoholic liver disease, fatty liver disease and hepatitis.

3. The method of claim 1, wherein the disorder is a psychiatric disorder selected from the group consisting of depressive disorders, eating disorders, bipolar disorder, abuse disorders, dependence disorders, Axis II disorders, psychosis and anxiety disorders.

4. The method of claim 3, wherein the psychiatric disorder is an anxiety disorder selected from the group consisting of generalized anxiety disorder, post traumatic stress disorder, panic disorder and obsessive compulsive disorder.

5. The method of claim 3, wherein the psychiatric disorder is a depressive disorder.

6. The method of claim 5, wherein the depressive disorder is major depressive disorder, minor depression, brief recurrent depression, dysthymia or depression NOS.

7. The method of claim 3, wherein the psychiatric disorder is an eating disorder selected from the group consisting of bulimia nervosa, anorexia nervosa, binge eating disorder, obesity, or eating disorder NOS.

8. The method of claim 3, wherein the psychiatric disorder is bipolar disorder, an abuse disorder or a dependence disorder.

9. The method of claim 8, wherein the psychiatric disorder includes abuse of, or dependence on, alcohol, cocaine, codeine, oxycodone, hydrocodone or other opiates.

10. The method of claim 3, wherein the psychiatric disorder is an Axis II disorder selected from borderline personality disorder.

11. The method of claim 1, wherein Tₘₐₓ is at least about 6 hours after administration of the extended release dosage.

12. The method of claim 1, wherein Tₘₐₓ is about 4 to about 12 hours after administration of the extended release dosage.

13. The method of claim 1, wherein the dose is administered in 1 to 4, 1 to 5 or 1 to 6 discrete dosage units.

14. The method of claim 1, wherein the patient is fed.

15. The method of claim 1, further comprising administering to the patient one or more additional active compounds.

16. The method of claim 15, wherein the one or more additional compounds comprise vitamin B12 (B12), folate (folic acid or a biologically acceptable salt thereof), or both.

17. The method of claim 1, wherein at least a portion of the SAMe is contained within an extended release matrix, an osmotic extended release core or a pulsatile release formulation.

18. An extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.95 when T is about 2 hours; Q is about 0.5 to about 1.0 when T is about 4 hours; Q is about 0.5 to about 1.0 when T is about 6 hours; Q is about 0.3 to about 0.9 when T is about 8 hours; and Q is about 0.15 to about 0.6 when T is about 12 hours.

19. A kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.95 when T is about 2 hours; Q is about 0.5 to about 1.0 when T is about 4 hours; Q is about 0.5 to about 1.0 when T is about 6 hours; Q is about 0.3 to about 0.9 when T is about 8 hours; and Q is about 0.15 to about 0.6 when T is about 12 hours.

20. The kit of claim 19, wherein the kit further comprises at least one dosage form selected from the group consisting of an immediate release SAMe dosage and an enterically coated immediate release SAMe dosage.

21. A method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.5 to about 0.95 when T is about 2 hours; Q is about 0.6 to about 0.95 when T is about 2 hours; Q is about 0.65 to about 0.95 when T is about 4 hours; Q is about 0.9 to about 1.0 when T is about 6 hours; Q is about 0.7 to about 0.95 when T is about 8 hours; and Q is about 0.3 to about 0.65 (especially about 0.5 to about 0.6) when T is about 12 hours.

22. The method of claim 21, wherein the disorder is a liver disorder selected from the group consisting of alcoholic liver disease, fatty liver disease and hepatitis.

23. The method of claim 21, wherein the disorder is a psychiatric disorder selected from the group consisting of depressive disorders, eating disorders, bipolar disorder, abuse disorders, dependence disorders, Axis II disorders, psychosis and anxiety disorders.

24. The method of claim 23, wherein the psychiatric disorder is an anxiety disorder selected from the group consisting of generalized anxiety disorder, post traumatic stress disorder, panic disorder and obsessive compulsive disorder.

25. The method of claim 23, wherein the psychiatric disorder is a depressive disorder.

26. The method of claim 25, wherein the depressive disorder is major depressive disorder, minor depression, brief recurrent depression, dysthymia or depression NOS.

27. The method of claim 23, wherein the psychiatric disorder is an eating disorder selected from the group consisting of bulimia nervosa, anorexia nervosa, binge eating disorder, obesity, or eating disorder NOS.

28. The method of claim 23, wherein the psychiatric disorder is bipolar disorder, an abuse disorder or a dependence disorder.

29. The method of claim 28, wherein the psychiatric disorder includes abuse of, or dependence on, alcohol, cocaine, codeine, oxycodone, hydrocodone or other opiates.

30. The method of claim 23, wherein the psychiatric disorder is an Axis II disorder selected from borderline personality disorder.

31. The method of claim 21, wherein Tₘₐₓ is at least about 6 hours after administration of the extended release dosage.

32. The method of claim 21, wherein Tₘₐₓ is about 4 to about 12 hours after administration of the extended release dosage.

33. The method of claim 21, wherein the dose is administered in 1 to 4, 1 to 5 or 1 to 6 discrete dosage units.

34. The method of claim 21, wherein the patient is fed.

35. The method of claim 21, further comprising administering to the patient one or more additional active compounds.

36. The method of claim 35, wherein the one or more additional compounds comprise vitamin B12 (B12), folate (folic acid or a biologically acceptable salt thereof), or both.

37. The method of claim 21, wherein at least a portion of the SAMe is contained within an extended release matrix, an osmotic extended release core or a pulsatile release formulation.

38. An extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.6 to about 0.95 when T is about 2 hours; Q is about 0.65 to about 0.95 when T is about 4 hours; Q is about 0.9 to about 1.0 when T is about 6 hours; Q is about 0.7 to about 0.95 when T is about 8 hours; and Q is about 0.3 to about 0.65 (especially about 0.5 to about 0.6) when T is about 12 hours.

39. A kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.6 to about 0.95 when T is about 2 hours; Q is about 0.65 to about 0.95 when T is about 4 hours; Q is about 0.9 to about 1.0 when T is about 6 hours; Q is about 0.7 to about 0.95 when T is about 8 hours; and Q is about 0.3 to about 0.65 (especially about 0.5 to about 0.6) when T is about 12 hours.

40. The kit of claim 39, wherein the kit further comprises at least one dosage form selected from the group consisting of an immediate release SAMe dosage and an enterically coated immediate release SAMe dosage.

41. A method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.7 to about 0.9 when T is about 2 hours; Q is about 0.7 to about 0.9 when T is about 4 hours; Q is about 0.9 to about 1.0 when T is about 6 hours; Q is about 0.4 to about 0.6 when T is about 8 hours; and Q is about 0.25 to about 0.45 when T is about 12 hours.

42. The method of claim 41, wherein the disorder is a liver disorder selected from the group consisting of alcoholic liver disease, fatty liver disease and hepatitis.

43. The method of claim 41, wherein the disorder is a psychiatric disorder selected from the group consisting of depressive disorders, eating disorders, bipolar disorder, abuse disorders, dependence disorders, Axis II disorders, psychosis and anxiety disorders.

44. The method of claim 43, wherein the psychiatric disorder is an anxiety disorder selected from the group consisting of generalized anxiety disorder, post traumatic stress disorder, panic disorder and obsessive compulsive disorder.

45. The method of claim 43, wherein the psychiatric disorder is a depressive disorder.

46. The method of claim 45, wherein the depressive disorder is major depressive disorder, minor depression, brief recurrent depression, dysthymia or depression NOS.

47. The method of claim 43, wherein the psychiatric disorder is an eating disorder selected from the group consisting of bulimia nervosa, anorexia nervosa, binge eating disorder, obesity, or eating disorder NOS.

48. The method of claim 43, wherein the psychiatric disorder is bipolar disorder, an abuse disorder or a dependence disorder.

49. The method of claim 48, wherein the psychiatric disorder includes abuse of, or dependence on, alcohol, cocaine, codeine, oxycodone, hydrocodone or other opiates.

50. The method of claim 43, wherein the psychiatric disorder is an Axis II disorder selected from borderline personality disorder.

51. The method of claim 41, wherein Tₘₐₓ is at least about 6 hours after administration of the extended release dosage.

52. The method of claim 41, wherein Tₘₐₓ is about 4 to about 12 hours after administration of the extended release dosage.

53. The method of claim 41, wherein the dose is administered in 1 to 4, 1 to 5 or 1 to 6 discrete dosage units.

54. The method of claim 41, wherein the patient is fed.

55. The method of claim 41, further comprising administering to the patient one or more additional active compounds.

56. The method of claim 45, wherein the one or more additional compounds comprise vitamin B12 (B12), folate (folic acid or a biologically acceptable salt thereof), or both.

57. The method of claim 41, wherein at least a portion of the SAMe is contained within an extended release matrix, an osmotic extended release core or a pulsatile release formulation.

58. An extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.7 to about 0.9 when T is about 2 hours; Q is about 0.7 to about 0.9 when T is about 4 hours; Q is about 0.9 to about 1.0 when T is about 6 hours; Q is about 0.4 to about 0.6 when T is about 8 hours; and Q is about 0.25 to about 0.45 when T is about 12 hours.

59. A kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.7 to about 0.9 when T is about 2 hours; Q is about 0.7 to about 0.9 when T is about 4 hours; Q is about 0.9 to about 1.0 when T is about 6 hours; Q is about 0.4 to about 0.6 when T is about 8 hours; and Q is about 0.25 to about 0.45 when T is about 12 hours.

60. The kit of claim 59, wherein the kit further comprises at least one dosage form selected from the group consisting of an immediate release SAMe dosage and an enterically coated immediate release SAMe dosage.

61. A method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.8 to about 1.0 when T is about 4 hours; Q is about 0.4 to about 0.8 when T is about 6 hours; Q is about 0.2 to about 0.7 when T is about 8 hours; and Q is about 0.2 to about 0.7 when T is about 12 hours.

62. The method of claim 61, wherein the disorder is a liver disorder selected from the group consisting of alcoholic liver disease, fatty liver disease and hepatitis.

63. The method of claim 61, wherein the disorder is a psychiatric disorder selected from the group consisting of depressive disorders, eating disorders, bipolar disorder, abuse disorders, dependence disorders, Axis II disorders, psychosis and anxiety disorders.

64. The method of claim 63, wherein the psychiatric disorder is an anxiety disorder selected from the group consisting of generalized anxiety disorder, post traumatic stress disorder, panic disorder and obsessive compulsive disorder.

65. The method of claim 63, wherein the psychiatric disorder is a depressive disorder.

66. The method of claim 65, wherein the depressive disorder is major depressive disorder, minor depression, brief recurrent depression, dysthymia or depression NOS.

67. The method of claim 63, wherein the psychiatric disorder is an eating disorder selected from the group consisting of bulimia nervosa, anorexia nervosa, binge eating disorder, obesity, or eating disorder NOS.

68. The method of claim 63, wherein the psychiatric disorder is bipolar disorder, an abuse disorder or a dependence disorder.

69. The method of claim 68, wherein the psychiatric disorder includes abuse of, or dependence on, alcohol, cocaine, codeine, oxycodone, hydrocodone or other opiates.

70. The method of claim 63, wherein the psychiatric disorder is an Axis II disorder selected from borderline personality disorder.

71. The method of claim 61, wherein Tₘₐₓ is at least about 6 hours after administration of the extended release dosage.

72. The method of claim 61, wherein Tₘₐₓ is about 4 to about 12 hours after administration of the extended release dosage.

73. The method of claim 61, wherein the dose is administered in 1 to 4, 1 to 5 or 1 to 6 discrete dosage units.

74. The method of claim 61, wherein the patient is fed.

75. The method of claim 61, further comprising administering to the patient one or more additional active compounds.

76. The method of claim 65, wherein the one or more additional compounds comprise vitamin B12 (B12), folate (folic acid or a biologically acceptable salt thereof), or both.

77. The method of claim 61, wherein at least a portion of the SAMe is contained within an extended release matrix, an osmotic extended release core or a pulsatile release formulation.

78. An extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.8 to about 1.0 when T is about 4 hours; Q is about 0.4 to about 0.8 when T is about 6 hours; Q is about 0.2 to about 0.7 when T is about 8 hours; and Q is about 0.2 to about 0.7 when T is about 12 hours.

79. A kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form comprising an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.8 to about 1.0 when T is about 4 hours; Q is about 0.4 to about 0.8 when T is about 6 hours; Q is about 0.2 to about 0.7 when T is about 8 hours; and Q is about 0.2 to about 0.7 when T is about 12 hours.

80. The kit of claim 79, wherein the kit further comprises at least one dosage form selected from the group consisting of an immediate release SAMe dosage and an enterically coated immediate release SAMe dosage.

81. A method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.5 to about 0.8 when T is about 2 hours; Q is about 0.8 to about 1.0 when T is about 4 hours; Q is about 0.8 to about 1.0 when T is about 6 hours; Q is about 0.3 to about 0.7 when T is about 8 hours; and Q is about 0.3 to about 0.7 when T is about 12 hours.

82. The method of claim 81, wherein the disorder is a liver disorder selected from the group consisting of alcoholic liver disease, fatty liver disease and hepatitis.

83. The method of claim 81, wherein the disorder is a psychiatric disorder selected from the group consisting of depressive disorders, eating disorders, bipolar disorder, abuse disorders, dependence disorders, Axis II disorders, psychosis and anxiety disorders.

84. The method of claim 83, wherein the psychiatric disorder is an anxiety disorder selected from the group consisting of generalized anxiety disorder, post traumatic stress disorder, panic disorder and obsessive compulsive disorder.

85. The method of claim 83, wherein the psychiatric disorder is a depressive disorder.

86. The method of claim 85, wherein the depressive disorder is major depressive disorder, minor depression, brief recurrent depression, dysthymia or depression NOS.

87. The method of claim 83, wherein the psychiatric disorder is an eating disorder selected from the group consisting of bulimia nervosa, anorexia nervosa, binge eating disorder, obesity, or eating disorder NOS.

88. The method of claim 83, wherein the psychiatric disorder is bipolar disorder, an abuse disorder or a dependence disorder.

89. The method of claim 88, wherein the psychiatric disorder includes abuse of, or dependence on, alcohol, cocaine, codeine, oxycodone, hydrocodone or other opiates.

90. The method of claim 83, wherein the psychiatric disorder is an Axis II disorder selected from borderline personality disorder.

91. The method of claim 81, wherein Tₘₐₓ is at least about 6 hours after administration of the extended release dosage.

92. The method of claim 81, wherein Tₘₐₓ is about 4 to about 12 hours after administration of the extended release dosage.

93. The method of claim 81, wherein the dose is administered in 1 to 4, 1 to 5 or 1 to 6 discrete dosage units.

94. The method of claim 81, wherein the patient is fed.

95. The method of claim 81, further comprising administering to the patient one or more additional active compounds.

96. The method of claim 85, wherein the one or more additional compounds comprise vitamin B12 (B12), folate (folic acid or a biologically acceptable salt thereof), or both.

97. The method of claim 81, wherein at least a portion of the SAMe is contained within an extended release matrix, an osmotic extended release core or a pulsatile release formulation.

98. An extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.5 to about 0.8 when T is about 2 hours; Q is about 0.8 to about 1.0 when T is about 4 hours; Q is about 0.8 to about 1.0 when T is about 6 hours; Q is about 0.3 to about 0.7 when T is about 8 hours; and Q is about 0.3 to about 0.7 when T is about 12 hours.

99. A kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form×release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.5 to about 0.8 when T is about 2 hours; Q is about 0.8 to about 1.0 when T is about 4 hours; Q is about 0.8 to about 1.0 when T is about 6 hours; Q is about 0.3 to about 0.7 when T is about 8 hours; and Q is about 0.3 to about 0.7 when T is about 12 hours.

100. The kit of claim 99, wherein the kit further comprises at least one dosage form selected from the group consisting of an immediate release SAMe dosage and an enterically coated immediate release SAMe dosage.

101. A method of treating a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising administering to the patient an extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.5 to about 0.7 when T is about 4 hours; Q is about 0.6 to about 0.8 when T is about 6 hours; Q is about 0.8 to about 1.0 when T is about 8 hours; and Q is about 0.5 to about 0.7 when T is about 12 hours.

102. The method of claim 81, wherein the disorder is a liver disorder selected from the group consisting of alcoholic liver disease, fatty liver disease and hepatitis.

103. The method of claim 81, wherein the disorder is a psychiatric disorder selected from the group consisting of depressive disorders, eating disorders, bipolar disorder, abuse disorders, dependence disorders, Axis II disorders, psychosis and anxiety disorders.

104. The method of claim 83, wherein the psychiatric disorder is an anxiety disorder selected from the group consisting of generalized anxiety disorder, post traumatic stress disorder, panic disorder and obsessive compulsive disorder.

105. The method of claim 83, wherein the psychiatric disorder is a depressive disorder.

106. The method of claim 85, wherein the depressive disorder is major depressive disorder, minor depression, brief recurrent depression, dysthymia or depression NOS.

107. The method of claim 83, wherein the psychiatric disorder is an eating disorder selected from the group consisting of bulimia nervosa, anorexia nervosa, binge eating disorder, obesity, or eating disorder NOS.

108. The method of claim 83, wherein the psychiatric disorder is bipolar disorder, an abuse disorder or a dependence disorder.

109. The method of claim 88, wherein the psychiatric disorder includes abuse of, or dependence on, alcohol, cocaine, codeine, oxycodone, hydrocodone or other opiates.

110. The method of claim 83, wherein the psychiatric disorder is an Axis II disorder selected from borderline personality disorder.

111. The method of claim 81, wherein Tₘₐₓ is at least about 6 hours after administration of the extended release dosage.

112. The method of claim 81, wherein Tₘₐₓ is about 4 to about 12 hours after administration of the extended release dosage.

113. The method of claim 81, wherein the dose is administered in 1 to 4, 1 to 5 or 1 to 6 discrete dosage units.

114. The method of claim 81, wherein the patient is fed.

115. The method of claim 81, further comprising administering to the patient one or more additional active compounds.

116. The method of claim 85, wherein the one or more additional compounds comprise vitamin B12 (B12), folate (folic acid or a biologically acceptable salt thereof), or both.

117. The method of claim 81, wherein at least a portion of the SAMe is contained within an extended release matrix, an osmotic extended release core or a pulsatile release formulation.

118. An extended release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.5 to about 0.7 when T is about 4 hours; Q is about 0.6 to about 0.8 when T is about 6 hours; Q is about 0.8 to about 1.0 when T is about 8 hours; and Q is about 0.5 to about 0.7 when T is about 12 hours.

119. A kit for treatment of a disorder selected from the group consisting of osteoarthritis, rheumatoid arthritis, fibromyalgia, a psychiatric disorder, an inflammatory condition, a central nervous system (CNS) disorder, a pain disorder and a liver disorder, in a patient, comprising at least one dosage form×release dosage comprising a therapeutically effective amount of SAMe, wherein the extended release dosage provides a quotient Q=(([SAMe]_{T}−[SAMe]₀)/Cₘₐₓ), wherein Cₘₐₓ=[SAMe]_{Max}−[SAMe]₀ and [SAMe]_{Max} is a maximum blood plasma concentration of SAMe in a patient population after administration of SAMe to the patient population, [SAMe]₀ is a blood plasma concentration of SAMe immediately prior to administration of SAMe to the patient population and [SAMe]_{T} is a blood plasma concentration of SAMe at time T after administration of SAMe to the patient population); Q is about 0.4 to about 0.6 when T is about 2 hours; Q is about 0.5 to about 0.7 when T is about 4 hours; Q is about 0.6 to about 0.8 when T is about 6 hours; Q is about 0.8 to about 1.0 when T is about 8 hours; and Q is about 0.5 to about 0.7 when T is about 12 hours.

120. The kit of claim 99, wherein the kit further comprises at least one dosage form selected from the group consisting of an immediate release SAMe dosage and an enterically coated immediate release SAMe dosage.

121. An extended release, oral dosage for administration of SAMe to a patient, comprising a therapeutically effective amount of SAMe, wherein dissolution of the oral dosage in a USP II dissolution apparatus in aqueous buffer having an initial pH of about 6.8 provides less about 70% release of SAMe after about 2 hours, less than about 80% release of SAMe after about 3 hours and less than about 100% release of SAMe after about 4 hours.

122. An extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours.

123. An extended release, oral dosage for administration of SAMe to a patient, comprising a therapeutically effective amount of SAMe, wherein dissolution of the oral dosage in a USP II dissolution apparatus in aqueous buffer at an initial pH of about 6.8 provides less about 70% release of SAMe after about 2 hours, less than about 80% release of SAMe after about 3 hours, less than about 100% release of SAMe after about 4 hours, and at least about 50% release after about 8 hours.

124. An extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours, and at least about 70% release after about 8 hours.

125. An extended release, oral dosage for administration of SAMe to a patient, comprising a therapeutically effective amount of SAMe, liquid paraffin, magnesium aluminometasilicate and 0-6% of an extended release coating, which optionally comprises a pore former.

126. A kit for administration of SAMe to a patient, comprising at least a first dosage form and a second dosage form, wherein said first dosage form is an immediate release dosage optionally comprising an enteric coating; and the second dosage form is an extended release dosage form.

127. The kit of claim 126, wherein the kit comprises an extended release, oral dosage for administration of SAMe to a patient, comprising a therapeutically effective amount of SAMe, wherein dissolution of the oral dosage in a USP II dissolution apparatus in aqueous buffer having an initial pH of about 6.8 provides less about 70% release of SAMe after about 2 hours, less than about 80% release of SAMe after about 3 hours and less than about 100% release of SAMe after about 4 hours.

128. The kit of claim 126, wherein the kit comprises an extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours.

129. The kit of claim 126, wherein the kit comprises an extended release, oral dosage for administration of SAMe to a patient, comprising a therapeutically effective amount of SAMe, wherein dissolution of the oral dosage in a USP II dissolution apparatus in aqueous buffer at an initial pH of about 6.8 provides less about 70% release of SAMe after about 2 hours, less than about 80% release of SAMe after about 3 hours, less than about 100% release of SAMe after about 4 hours, and at least about 50% release after about 8 hours.

130. The kit of claim 126, wherein the kit comprises an extended release, oral dosage for administration of SAMe to a patient, comprising a therapeutically effective amount of SAMe, wherein the oral dosage is not enterically coated, and wherein dissolution of the oral dosage in a USP II dissolution apparatus in aqueous HCl having an initial pH of about 1 provides less about 70% release of SAMe after about 2 hours, less than about 80% release of SAMe after about 3 hours and less than about 100% release of SAMe after about 4 hours, and at least about 70% release after about 8 hours.

131. The kit of claim 126, wherein the kit comprises an extended release, oral dosage for administration of SAMe to a patient, comprising a therapeutically effective amount of SAMe, liquid paraffin, magnesium aluminometasilicate and 0-6% of an extended release coating, which optionally comprises a pore former.
